SEQUENCE LISTING (13 sequences)

SEQ ID NO: 1 - protein, 753 aa, Artificial Sequence
  Synthetic Polypeptide
MERRAWSLQCTAFVLFCAWCALNSAKAKRQFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRFGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHGTSSQPEHMKQPRVYTSYNTVQNDRRGVEKMVDPGEEQPTQENPKENTLVSKSPSSSSVGGRRDELEEGAPSQAMLRLLQSAFSKNSPPKQSPKKSPSAKLNIPYENFYEALEKLNKPSQLKDSEDSLYNDYVDVFYNTKPYKHRDDRLLQALVDILNEEN

SEQ ID NO: 2 - protein, 619 aa, Artificial Sequence
  Synthetic Polypeptide
MVFASAERPVFTNHFLVELHKGGEDKARQVAAEHGFGVRKLPFAEGLYHFYHNGLAKAKRRRSLHHKQQLERDPRVKMALQQEGFDRKKRGYRDINEIDINMNDPLFTKQWYLINTGQADGTPGLDLNVAEAWELGYTGKGVTIGIMDDGIDYLHPDLASNYNAEASYDFSSNDPYPYPRYTDDWFNSHGTRCAGEVSAAANNNICGVGVAYNSKVAGIRMLDQPFMTDIIEASSISHMPQLIDIYSASWGPTDNGKTVDGPRELTLQAMADGVNKGRGGKGSIYVWASGDGGSYDDCNCDGYASSMWTISINSAINDGRTALYDESCSSTLASTFSNGRKRNPEAGVATTDLYGNCTLRHSGTSAAAPEAAGVFALALEANLGLTWRDMQHLTVLTSKRNQLHDEVHQWRRNGVGLEFNHLFGYGVLDAGAMVKMAKDWKTVPERFHCVGGSVQDPEKIPSTGKLVLTLTTDACEGKENFVRYLEHVQAVITVNATRRGDLNINMTSPMGTKSILLSRRPRDDDSKVGFDKWPFMTTHTWGEDARGTWTLELGFVGSAPQKGVLKEWTLMLHGTQSAPYIDQVVRDYQSKLAMSKKEELEEELDEAVERSLKSILNKN

SEQ ID NO: 3 - protein, 794 aa, Artificial Sequence
  Synthetic Polypeptide
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

SEQ ID NO: 4 - protein, 755 aa, Artificial Sequence
  Synthetic Polypeptide
MRPAPIALWLRLVLALALVRPRAVGWAPVRAPIYVSSWAVQVSQGNREVERLARKFGFVNLGPIFPDGQYFHLRHRGVVQQSLTPHWGHRLHLKKNPKVQWFQQQTLQRRVKRSVVVPTDPWFSKQWYMNSEAQPDLSILQAWSQGLSGQGIVVSVLDDGIEKDHPDLWANYDPLASYDFNDYDPDPQPRYTPSKENRHGTRCAGEVAAMANNGFCGVGVAFNARIGGVRMLDGTITDVIEAQSLSLQPQHIHIYSASWGPEDDGRTVDGPGILTREAFRRGVTKGRGGLGTLFIWASGNGGLHYDNCNCDGYTNSIHTLSVGSTTQQGRVPWYSEACASTLTTTYSSGVATDPQIVTTDLHHGCTDQHTGTSASAPLAAGMIALALEANPFLTWRDMQHLVVRASKPAHLQAEDWRTNGVGRQVSHHYGYGLLDAGLLVDTARTWLPTQPQRKCAVRVQSRPTPILPLIYIRENVSACAGLHNSIRSLEHVQAQLTLSYSRRGDLEISLTSPMGTRSTLVAIRPLDVSTEGYNNWVFMSTHFWDENPQGVWTLGLENKGYYFNTGTLYRYTLLLYGTAEDMTARPTGPQVTSSACVQRDTEGLCQACDGPAYILGQLCLAYCPPRFFNHTRLVTAGPGHTAAPALRVCSSCHASCYTCRGGSPRDCTSCPPSSTLDQQQGSCMGPTTPDSRPRLRAAACPHHRCPASAMVLSLLAVTLGGPVLCGMSMDLPLYAWLSRARATPTKPQVWLPAGT

SEQ ID NO: 5 - protein, 913 aa, Artificial Sequence
  Synthetic Polypeptide
MGWGSRCCCPGRLDLLCVLALLGGCLLPVCRTRVYTNHWAVKIAGGFPEANRIASKYGFINIGQIGALKDYYHFYHSRTIKRSVISSRGTHSFISMEPKVEWIQQQVVKKRTKRDYDFSRAQSTYFNDPKWPSMWYMHCSDNTHPCQSDMNIEGAWKRGYTGKNIVVTILDDGIERTHPDLMQNYDALASCDVNGNDLDPMPRYDASNENKHGTRCAGEVAAAANNSHCTVGIAFNAKIGGVRMLDGDVTDMVEAKSVSFNPQHVHIYSASWGPDDDGKTVDGPAPLTRQAFENGVRMGRRGLGSVFVWASGNGGRSKDHCSCDGYTNSIYTISISSTAESGKKPWYLEECSSTLATTYSSGESYDKKIITTDLRQRCTDNHTGTSASAPMAAGIIALALEANPFLTWRDVQHVIVRTSRAGHLNANDWKTNAAGFKVSHLYGFGLMDAEAMVMEAEKWTTVPRQHVCVESTDRQIKTIRPNSAVRSIYKASGCSDNPNRHVNYLEHVVVRITITHPRRGDLAIYLTSPSGTRSQLLANRLFDHSMEGFKNWEFMTIHCWGERAAGDWVLEVYDTPSQLRNFKTPGKLKEWSLVLYGTSVQPYSPTNEFPKVERFRYSRVEDPTDDYGTEDYAGPCDPECSEVGCDGPGPDHCNDCLHYYYKLKNNTRICVSSCPPGHYHADKKRCRKCAPNCESCFGSHGDQCMSCKYGYFLNEETNSCVTHCPDGSYQDTKKNLCRKCSENCKTCTEFHNCTECRDGLSLQGSRCSVSCEDGRYFNGQDCQPCHRFCATCAGAGADGCINCTEGYFMEDGRCVQSCSISYYFDHSSENGYKSCKKCDISCLTCNGPGFKNCTSCPSGYLLDLGMCQMGAICKDATEESWAEGGFCMLVKKNNLCQRKVLQQLCCKTCTFQG

SEQ ID NO: 6 - protein, 895 aa, Artificial Sequence
  Synthetic Polypeptide
MPPRAPPAPGPRPPPRAAAATDTAAGAGGAGGAGGAGGPGFRPLAPRPWRWLLLLALPAACSAPPPRPVYTNHWAVQVLGGPAEADRVAAAHGYLNLGQIGNLEDYYHFYHSKTFKRSTLSSRGPHTFLRMDPQVKWLQQQEVKRRVKRQVRSDPQALYFNDPIWSNMWYLHCGDKNSRCRSEMNVQAAWKRGYTGKNVVVTILDDGIERNHPDLAPNYDSYASYDVNGNDYDPSPRYDASNENKHGTRCAGEVAASANNSYCIVGIAYNAKIGGIRMLDGDVTDVVEAKSLGIRPNYIDIYSASWGPDDDGKTVDGPGRLAKQAFEYGIKKGRQGLGSIFVWASGNGGREGDYCSCDGYTNSIYTISVSSATENGYKPWYLEECASTLATTYSSGAFYERKIVTTDLRQRCTDGHTGTSVSAPMVAGIIALALEAKSIPLVQVLRTTALTSACAEHSDQRVVYLEHVVVRTSISHPRRGDLQIYLVSPSGTKSQLLAKRLLDLSNEGFTNWEFMTVHCWGEKAEGQWTLEIQDLPSQVRNPEKQGKLKEWSLILYGTAEHPYHTFSAHQSRSRMLELSAPELEPPKAALSPSQVEVPEDEEDYTAQSTPGSANILQTSVCHPECGDKGCDGPNADQCLNCVHFSLGSVKTSRKCVSVCPLGYFGDTAARRCRRCHKGCETCSSRAATQCLSCRRGFYHHQEMNTCVTLCPAGFYADESQKNCLKCHPSCKKCVDEPEKCTVCKEGFSLARGSCIPDCEPGTYFDSELIRCGECHHTCGTCVGPGREECIHCAKNFHFHDWKCVPACGEGFYPEEMPGLPHKVCRRCDENCLSCAGSSRNCSRCKTGFTQLGTSCITNHTCSNADETFCEMVKSNRLCERKLFIQFCCRTCLLAG

SEQ ID NO: 7 - protein, 785 aa, Artificial Sequence
  Synthetic Polypeptide
MPKGRQKVPHLDAPLGLPTCLWLELAGLFLLVPWVMGLAGTGGPDGQGTGGPSWAVHLESLEGDGEEETLEQQADALAQAAGLVNAGRIGELQGHYLFVQPAGHRPALEVEAIRQQVEAVLAGHEAVRWHSEQRLLRRAKRSVHFNDPKYPQQWHLNNRRSPGRDINVTGVWERNVTGRGVTVVVVDDGVEHTIQDIAPNYSPEGSYDLNSNDPDPMPHPDVENGNHHGTRCAGEIAAVPNNSFCAVGVAYGSRIAGIRVLDGPLTDSMEAVAFNKHYQINDIYSCSWGPDDDGKTVDGPHQLGKAALQHGVIAGRQGFGSIFVVASGNGGQHNDNCNYDGYANSIYTVTIGAVDEEGRMPFYAEECASMLAVTFSGGDKMLRSIVTTDWDLQKGTGCTEGHTGTSAAAPLAAGMIALMLQVRPCLTWRDVQHIIVFTATRYEDRRAEWVTNEAGFSHSHQHGFGLLNAWRLVNAAKIWTSVPYLASYVSPVLKENKAIPQSPRSLEVLWNVSRMDLEMSGLKTLEHVAVTVSITHPRRGSLELKLFCPSGMMSLIGAPRSMDSDPNGFNDWTFSTVRCWGERARGTYRLVIRDVGDESFQVGILRQWQLTLYGSVWSAVDIRDRQRLLESAMSGKYLHDDFALPCPPGLKIPEEDGYTITPNTLKTLVLVGCFTVFWTVYYMLEVYLSQRNVASNQVCRSGPCHWPHRSRKAKEEGTELESVPLCSSKDPDEVETESRGPPTTSDLLAPDLLEQGDWSLSQNKSALDCPHQHLDVPHGKEEQIC

SEQ ID NO: 8 - protein, 1052 aa, Artificial Sequence
  Synthetic Polypeptide
MKLVNIWLLLLVVLLCGKKHLGDRLEKKSFEKAPCPGCSHLTLKVEFSSTVVEYEYIVAFNGYFTAKARNSFISSALKSSEVDNWRIIPRNNPSSDYPSDFEVIQIKEKQKAGLLTLEDHPNIKRVTPQRKVFRSLKYAESDPTVPCNETRWSQKWQSSRPLRRASLSLGSGFWHATGRHSSRRLLRAIPRQVAQTLQADVLWQMGYTGANVRVAVFDTGLSEKHPHFKNVKERTNWTNERTLDDGLGHGTFVAGVIASMRECQGFAPDAELHIFRVFTNNQVSYTSWFLDAFNYAILKKIDVLNLSIGGPDFMDHPFVDKVWELTANNVIMVSAIGNDGPLYGTLNNPADQMDVIGVGGIDFEDNIARFSSRGMTTWELPGGYGRMKPDIVTYGAGVRGSGVKGGCRALSGTSVASPVVAGAVTLLVSTVQKRELVNPASMKQALIASARRLPGVNMFEQGHGKLDLLRAYQILNSYKPQASLSPSYIDLTECPYMWPYCSQPIYYGGMPTVVNVTILNGMGVTGRIVDKPDWQPYLPQNGDNIEVAFSYSSVLWPWSGYLAISISVTKKAASWEGIAQGHVMITVASPAETESKNGAEQTSTVKLPIKVKIIPTPPRSKRVLWDQYHNLRYPPGYFPRDNLRMKNDPLDWNGDHIHTNFRDMYQHLRSMGYFVEVLGAPFTCFDASQYGTLLMVDSEEEYFPEEIAKLRRDVDNGLSLVIFSDWYNTSVMRKVKFYDENTRQWWMPDTGGANIPALNELLSVWNMGFSDGLYEGEFTLANHDMYYASGCSIAKFPEDGVVITQTFKDQGLEVLKQETAVVENVPILGLYQIPAEGGGRIVLYGDSNCLDDSHRQKDCFWLLDALLQYTSYGVTPPSLSHSGNRQRPPSGAGSVTPERMEGNHLHRYSKVLEAHLGDPKPRPLPACPRLSWAKPQPLNETAPSNLWKHQKLLSIDLDKVVLPNFRSNRPQVRPLSPGESGAWDIPGGIMPGRYNQEVGQTIPVFAFLGAMVVLAFFVVQINKAKSRPKRRKPRVKRPQLMQQVHPPKTPSV

SEQ ID NO: 9 - protein, 692 aa, Artificial Sequence
  Synthetic Polypeptide
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 10 - protein, 381 aa, Bacillus subtilis
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 11 - protein, 814 aa, Saccharomyces cerevisiae
MKVRKYITLCFWWAFSTSALVSSQQIPLKDHTSRQYFAVESNETLSRLEEMHPNWKYEHDVRGLPNHYVFSKELLKLGKRSSLEELQGDNNDHILSVHDLFPRNDLFKRLPVPAPPMDSSLLPVKEAEDKLSINDPLFERQWHLVNPSFPGSDINVLDLWYNNITGAGVVAAIVDDGLDYENEDLKDNFCAEGSWDFNDNTNLPKPRLSDDYHGTRCAGEIAAKKGNNFCGVGVGYNAKISGIRILSGDITTEDEAASLIYGLDVNDIYSCSWGPADDGRHLQGPSDLVKKALVKGVTEGRDSKGAIYVFASGNGGTRGDNCNYDGYTNSIYSITIGAIDHKDLHPPYSEGCSAVMAVTYSSGSGEYIHSSDINGRCSNSHGGTSAAAPLAAGVYTLLLEANPNLTWRDVQYLSILSAVGLEKNADGDWRDSAMGKKYSHRYGFGKIDAHKLIEMSKTWENVNAQTWFYLPTLYVSQSTNSTEETLESVITISEKSLQDANFKRIEHVTVTVDIDTEIRGTTTVDLISPAGIISNLGVVRPRDVSSEGFKDWTFMSVAHWGENGVGDWKIKVKTTENGHRIDFHSWRLKLFGESIDSSKTETFVFGNDKEEVEPAATESTVSQYSASSTSISISATSTSSISIGVETSAIPQTTTASTDPDSDPNTPKKLSSPRQAMHYFLTIFLIGATFLVLYFMFFMKSRRRIRRSRAETYEFDIIDTDSEYDSTLDNGTSGITEPEEVEDFDFDLSDEDHLASLSSSENGDAEHTIDSVLTNENPFSDPIKQKFPNDANAESASNKLQELQPDVPPSSGRS

SEQ ID NO: 12 - protein, 4 aa, Artificial Sequence
  Synthetic Polypeptide
  misc_feature (2)..(2): Xaa can be any naturally occurring amino acid
  MISC_FEATURE (3)..(3): Xaa is Arg or Lys
RXXR

SEQ ID NO: 13 - protein, 4 aa, Artificial Sequence
  Synthetic Polypeptide
RVRR

(19) United States
(12) Patent Application Publication
(10) Pub. No.: US 2018/0171362 A1
(43) Pub. Date: Jun. 21, 2018

(54) ASSAYS FOR EVALUATING CELL CULTURE REAGENTS

(71) Applicant: Biogen MA Inc., Cambridge, MA (US)

(72) Inventors: Jessica Mondia, Medford, MA (US); Alan Gilbert, Arlington, MA (US); Maureen Lanan, Newton, MA (US)

(73) Assignee: Biogen MA Inc., Cambridge, MA (US)

(21) Appl. No.: 15/578,736

(22) PCT Filed: Jun. 1, 2016
(86) PCT No.: PCT/US2016/035355

§ 371 Date: Dec. 1, 2017

Related U.S. Application Data
(60) Provisional application No. 62/168,963, filed on Jun. 1, 2015.

(51) Int. Cl. C12P 1/00; C12Q 1/37; G01N 33/533

(52) U.S. Cl. CPC C12P 1/00; C12Q 1/37; C12Y 304/21094; G01N 2333/95; C12Y 304/21093; G01N 33/533

(57) ABSTRACT

Some aspects of the present disclosure provide methods, compositions and kits for identifying one or more reagents, such as a culture medium, that are effective for recombinant protein production.

### RELATED APPLICATIONS

This application claims the benefit under 35 U.S.C. § 119(e) of U.S. provisional patent application Ser. No. 62/168,963, filed Jun. 1, 2015, which is incorporated by reference herein in its entirety.

### FIELD OF THE INVENTION

Some aspects of the present disclosure relate generally to the field of biotechnology and more specifically to the field of recombinant protein technology.

### BACKGROUND

The ability to obtain consistently high yields of recombinant protein from cell cultures is important for efficient large scale production of biological products such as therapeutic proteins. However, many different factors can affect the reproducibility of cell growth and protein production in bioreactors. Minor variations in one or more known factors (including, for example, temperature, pH, oxygen, carbon dioxide, and nutrient levels) can significantly reduce levels of recombinant protein production. Nonetheless, despite the use of high quality reagents and efforts to carefully control growth conditions, large scale protein production often suffers from variable or inconsistent yields due to unknown factors.

### SUMMARY

Aspects of the disclosure relate to cell culture and protein production methods that involve evaluating one or more reagents (e.g., growth medium) for the presence or absence of proprotein convertase inhibitory activity. Surprisingly, certain lots of high grade growth medium were found to contain one or more inhibitors that reduced the yield of certain recombinant proteins produced in cell cultures (e.g., recombinant proteins that are processed by a proprotein convertase). Even more surprisingly, a rapid assay can be performed on cell culture reagents to predict whether they will support effective production of certain recombinant proteins from cells grown in bioreactors. In some embodiments, an assay comprises determining the presence or absence of a proprotein convertase inhibitor in a cell culture reagent prior to using the reagent for cell growth and/or protein expression. In some embodiments, an assay comprises determining a level (e.g., a relative level) of a proprotein convertase inhibitor in a cell culture reagent prior to using the reagent for cell growth and/or protein expression. In some embodiments, the level of proprotein convertase inhibitor in a cell culture reagent is predictive of the yield of correctly processed recombinant proteins from recombinant cells grown in the presence of the reagent. Accordingly, in some embodiments a cell culture reagent is discarded if it contains an unsatisfactory level of proprotein convertase inhibitor, and in some embodiments a cell culture reagent is used for cell growth and recombinant protein production if it contains an acceptably low level of proprotein convertase inhibitor.

Many newly synthesized recombinant proteins are activated only upon enzymatic cleavage of certain amino acids that block their activity. This processing from an inactive isoform to an active isoform is often mediated by enzymes of the proprotein convertase family. Efficiency of proprotein convertase cleavage can impact the yield of active recombinant protein isoforms.

The present disclosure is based, in part, on unexpected results showing that certain protein-free cell culture and protein synthesis reagents, including those formulated specifically to provide for high performance and consistency, in fact, contain contaminants that inhibit recombinant protein processing. Particularly surprising was data showing that the degree of contamination varies among different batches or lots of protein-free cell culture medium. Provided herein, in some aspects, are methods, compositions and kits for selecting reagents, such as culture medium, effective for recombinant protein synthesis, particularly those containing relatively low concentrations of enzymatic inhibitors.

Some aspects of the present disclosure provide assays for screening protein-free cell culture and protein synthesis reagents for inhibitors of proprotein convertase activity. Such assays, in some embodiments, utilize a probe that contains a proprotein convertase recognition site and emits a detectable signal only when cleaved by a cognate proprotein convertase. In some embodiments, a signal emitted from the probe in the presence of protein-free cell culture medium correlates inversely with a concentration of inhibitor (e.g., enzymatic contaminant). In some embodiments, a relatively high signal emitted from the probe in the presence of protein-free cell culture medium correlates with a low concentration of inhibitor (e.g., enzymatic contaminant). By contrast, in some embodiments, a relatively low signal emitted from the probe correlates with a high concentration of inhibitor (e.g., enzymatic contaminant). In some embodiments, the level of activity is compared to a known reference level. For example, the known reference level can be a signal emitted from the probe corresponding to a level of activity in the presence of a batch of cell culture medium that is suitable for production of a recombinant protein (e.g., a cell culture medium with a low concentration of enzymatic contaminant). In some embodiments, the level of activity in the presence of a cell culture reagent is compared to a reference level of activity obtained in the presence of an aqueous solution, buffer or other reagent that does not inhibit a proprotein convertase.

Some aspects of the present disclosure provide methods of identifying a reagent for use in the production of a recombinant protein, the methods comprising performing a proprotein convertase substrate assay on a sample of reagent (e.g., cell culture medium). In some aspects, performing a proprotein convertase substrate assay includes the steps of combining a sample of a reagent (e.g., a sample obtained from a cell culture medium) with a probe having a proprotein convertase recognition site that emits a detectable signal when cleaved at the recognition site, and a cognate proprotein convertase, thereby forming a mixture. The mixture may be incubated under conditions that result in cleavage of the probe at the proprotein convertase recognition site, which may be detected by performing a signal detection assay on the mixture.

In some embodiments, a signal from the mixture is detected as a result of performing a signal detection assay, for example, a fluorescence signal detection assay. The amount or intensity of the signal detected in the mixture, in some embodiments, is proportional to the amount of activity of the cognate proprotein convertase in the sample of the reagent. In some embodiments, the detectable signal from the mixture is a fluorescent signal.

In some embodiments, a proprotein convertase recognition site of the present disclosure comprises 2 or more (e.g., 3, 4, 5, 6, 7, 8, 9, 10, 11, 12, 13, 14, 15, 16, 17, 18, 19, or 20) amino acids of a PACE (Paired basic Amino acid Cleaving Enzyme) cleavage site (e.g., see Tian S, Biochemistry Insights, 2009:2, 9-20, incorporated by reference herein).

In some embodiments, a proprotein convertase recognition site of the present disclosure has the amino acid consensus sequence R-X-X-R, wherein R is arginine and X is any amino acid. In some embodiments, the proprotein convertase recognition site comprises the amino acid consensus sequence: R-X-(K/R)-R (SEQ ID NO: 12), wherein R is arginine, X is any amino acid, and K is lysine. In some embodiments, the proprotein convertase recognition site has the amino acid sequence: R-V-R-R (SEQ ID NO: 13), wherein R is arginine, and V is valine.

In some embodiments, the proprotein convertase, used in accordance with any of the methods described herein, is PCSK1, PCSK2, PCSK3/furin, PCSK4, PCSK5, PCSK6, PCSK7 or Kex2. In some embodiments, the proprotein convertase is PCSK3/furin.

In some embodiments, the reagent used in accordance with any of the methods, described herein, is a cell culture medium. In some embodiments, a proprotein convertase substrate assay is performed using a sample of a reagent. In some embodiments, the sample of the reagent is cell-free. In some embodiments, the sample of the reagent is protein-free.

In some embodiments, a probe with a proprotein convertase recognition site and a detectable molecule (e.g., a fluorophore) is used for detecting the activity of a proprotein convertase. In some embodiments, the probe has a protecting group linked to a fluorophore via a linker comprising the proprotein convertase recognition site. For example, the protecting group may be a tert-butyloxycarbonyl (t-Boc) protecting group or a 9-fluorenylmethyloxycarbonyl (Fmoc) protecting group. The fluorophore may be any suitable fluorophore capable of being detected in a proprotein convertase substrate assay. For example, the fluorophore may be 7-Amino-4-methylcoumarin (AMC). In some embodiments, the probe comprises a t-Boc protecting group linked to AMC via a linker that comprises the amino acid sequence: R-V-R-R (SEQ ID NO: 13), wherein R is arginine, and V is valine.

Any of the methods described herein may further include comparing a signal detected in a first mixture (e.g., a mixture obtained from a proprotein convertase substrate assay) containing a first sample of reagent, to a signal detected in a second mixture, containing a second sample of reagent. In some embodiments, the methods may include combining the first and second samples of reagent with a probe having a proprotein convertase recognition site that emits a detectable signal when cleaved at the recognition site, and the cognate proprotein convertase, thereby producing a first and a second mixture and incubating the mixtures under conditions that result in cleavage of the probe in the mixtures.

In some embodiments, the first sample of reagent and the second sample of reagent are of the same type and are obtained from separate lots.

In some embodiments a signal detection assay is performed on the first mixture and the second mixture.

In some embodiments, the methods further include detecting a signal in the mixtures as a result of performing the signal detection assay. The amount of signal detected in the first and second mixtures, in some embodiments, is proportional to the amount of activity of the cognate proprotein convertase in the first and second samples of reagent.

Accordingly, in some embodiments, a method of promoting recombinant protein yield from a recombinant cell culture in a bioreactor comprises performing an assay to determine a level of an inhibitor of proprotein convertase activity in one or more cell culture reagents, and using a cell culture reagent to support growth of the recombinant cell culture in the bioreactor only if the cell culture reagent is determined to contain a proprotein convertase inhibitor in an amount that is acceptable for the recombinant protein yield.

In some embodiments, the assay is a proprotein convertase substrate assay comprising (a) combining a sample of reagent with (i) a probe that comprises a proprotein convertase recognition site and emits a detectable signal when cleaved at the recognition site, and (ii) a cognate proprotein convertase, thereby forming a mixture; and (b) incubating the mixture under conditions that result in cleavage of the probe at the proprotein convertase recognition site. In some embodiments, a signal detection assay is performed on the mixture. In some embodiments, a signal is detected in the mixture as a result of performing the signal detection assay, and the amount of signal detected in the mixture is proportional to the amount of activity of the cognate proprotein convertase in the sample of the reagent. In some embodiments, the detectable signal is a fluorescent signal. In some embodiments, the proprotein convertase recognition site comprises 2 or more amino acids of a PACE cleavage site. In some embodiments, the proprotein convertase recognition site comprises the following amino acid consensus sequence: R-X-X-R, wherein R is arginine and X is any amino acid. In some embodiments, the proprotein convertase recognition site comprises the following amino acid consensus sequence: R-X-(K/R)-R (SEQ ID NO: 12), wherein R is arginine, X is any amino acid, and K is lysine. In some embodiments, the proprotein convertase recognition site comprises the following amino acid sequence: R-V-R-R (SEQ ID NO: 13), wherein R is arginine, and V is valine. In some embodiments, the proprotein convertase is selected from the group consisting of: PCSK1, PCSK2, PCSK3/furin, PCSK4, PCSK5, PCSK6, PCSK7 and Kex2. In some embodiments, the proprotein convertase is PCSK3/furin.

In some embodiments, the reagent is a powdered or liquid cell culture medium. In some embodiments, the sample of the reagent is cell-free. In some embodiments, the sample of the reagent is protein-free.

In some embodiments, the probe comprises a protecting group linked to a fluorophore via a linker comprising the proprotein convertase recognition site. In some embodiments, the protecting group is a tert-butyloxycarbonyl (t-Boc) protecting group. In some embodiments, the protecting group is a 9-fluorenylmethyloxycarbonyl (Fmoc) protecting group. In some embodiments, the fluorophore is 7-Amino-4-methylcoumarin (AMC). In some embodiments, the probe comprises a t-Boc protecting group linked to AMC via a linker that comprises the following amino acid sequence: R-V-R-R (SEQ ID NO: 13), wherein R is arginine, and V is valine.

In some embodiments, an assay also comprises comparing a signal detected in a mixture comprising a first sample of reagent to a signal detected in a separate mixture comprising a second sample of reagent. In some embodiments, the separate mixture is produced by combining the second sample of reagent with (i) a probe that comprises the proprotein convertase recognition site and emits a detectable signal when cleaved at the recognition site, and (ii) a cognate proprotein convertase, thereby producing the separate mixture; and incubating the separate mixture under conditions that result in cleavage of the probe of step.

In some embodiments, the sample of the first reagent and the sample of the second reagent are of the same type and are obtained from separate lots. In some embodiments, a signal detection assay is performed on the separate mixture. In some embodiments, the amount of signal detected in the separate mixture is proportional to the amount of activity of the cognate proprotein convertase in the second sample of reagent. In some embodiments, a reagent is selected (e.g., to be used in a protein production procedure) based on the amount of signal detected in each of the mixtures (e.g., by comparing the amount of signal detected in the assay for the mixture comprising the first sample of reagent to the amount of signal detected in the assay for the mixture comprising the second sample of reagent). In some embodiments, the reagent that is selected is the one that contains lower levels of proprotein convertase inhibitor.

Accordingly, in some embodiments, a method of selecting a reagent for use in the production of a recombinant protein comprises performing a proprotein convertase substrate assay in the presence of a sample of a reagent; determining a level of activity of a proprotein convertase and/or a level of proprotein convertase inhibitor; and identifying the reagent as acceptable for use in a recombinant cell culture to produce the recombinant protein if the level of activity of the proprotein convertase is at or above a threshold level sufficient for recombinant protein production (e.g., if the level of proprotein convertase inhibitor is sufficiently low to be suitable for recombinant protein production).

In some embodiments, the level of activity of the proprotein convertase is determined relative to the level of activity of a second proprotein convertase in the presence of a second sample of reagent that does not inhibit the activity of the second proprotein convertase (or that inhibits the activity of the second proprotein convertase at a sufficiently low level to be suitable for recombinant protein production). In some embodiments, the threshold level is the level of activity of the second proprotein convertase. In some embodiments, threshold level is 95%, 90%, 85%, 80%, 70%, 60%, 50%, 40%, 30%, 20%, or 10% of the level of activity of the second proprotein convertase.

In some embodiments, the methods further comprise selecting one of the reagents for use in cell culture and/or protein production in a bioreactor. In some embodiments, the selected reagent is the one that has the lower amount of proprotein convertase inhibitory activity.

### BRIEF DESCRIPTION OF DRAWINGS

FIG. 1A is a graph showing non-processed isoform (NPI) values as a function of CD OptiCHO™ Lots. Lots of OptiCHO™ which produce high NPI demonstrate furin inhibition more than lots with low NPI; FIG. 1B shows both a schematic of a NPI and processed isoform (PI).

FIG. 2 is a non-limiting schematic representation of the fluorometric assay developed to test furin inhibition in the presence of a reagent.

FIG. 3 is a graph showing theoretical picomolar amounts of 7 amino-4-methyl coumarin (AMC) released as a function of time in minutes. The amount of AMC released over time is decreased with inhibition of furin, as depicted by the arrow.

FIGS. 4A-4B are graphs showing furin activity for 6 different lots of OptiCHO™ medium. FIG. 4A is a graph showing fluorescence intensity (RFU) values as a function of time in minutes. FIG. 4B is a graph showing the rate of furin activity (RFU/min) as a function of OptiCHO™ lots. The results are an average of five independent experiments.

FIG. 5 is a graph showing the rate of furin activity (RFU/min) as a function NPI.

FIGS. 6A-6B are bar graphs of furin activity (RFU/min) in PBS, in OptiCHO™ medium Lot 1, OptiCHO™ medium Lot 2, OptiCHO™ medium Lot 3. The furin activity was tested on different dates to measure intermediate precision.

FIG. 7 is a graph showing the rate of furin activity (RFU/min) of OptiCHO™, which produced low NPI as a function of various degradation factors including room temperature (no light), light (1 W/m²) and an oven (˜40° C.). <Condition><Moisture Content>-<days>

FIG. 8 is a non-limiting schematic representation of a probe having two fluorophores that produce a FRET signal. Upon cleavage of the probe at a proprotein convertase recognition site by a proprotein convertase, the FRET signal is not produced.

FIG. 9 is a non-limiting schematic representation of a probe having a fluorophore and a quenching molecule. The quenching molecule absorbs the fluorescent signal from the fluorophore. Upon cleavage of the probe at a proprotein convertase recognition site by a proprotein convertase the fluorophore and quenching molecule are separated, allowing the signal from the fluorophore to be detected. (fluor=fluorophore; quench=quenching molecule; PCRS=proprotein convertase recognition site.)

FIG. 10 shows that spent samples from a small scale bioreactor also show the same inhibition as the cell culture media prior to use.

### DETAILED DESCRIPTION

Provided herein are methods and compositions for improving the yield and reproducibility of protein production techniques using recombinant cells in bioreactors. In some aspects, methods for screening growth medium are provided to detect one or more factors that can reduce the yield of certain recombinant proteins obtained from cell cultures. The methods include, in some embodiments, a rapid assay that can be performed on reagent material to predict whether the material will support effective production of certain recombinant proteins from cells grown in bioreactors. For example, the methods described herein can include an assay for determining the level of one or more proprotein convertase inhibitors in growth medium. In some embodiments, the level of inhibitors in growth medium is indicative of the yield of appropriately processed proteins obtained from recombinant cells grown in the medium.

Provided herein, in some aspects, are methods, compositions and kits for identifying reagents, such as culture medium, effective for recombinant protein synthesis, in particular those reagents containing low or acceptable concentrations of enzymatic inhibitors.

Identifying a reagent for use in the production of a recombinant protein, in some aspects, includes performing a proprotein convertase substrate assay on a sample of reagent. Prior to producing a recombinant protein, for example a protein that is activated upon cleavage by a proprotein convertase, a reagent used in the production process (e.g., a cell culture medium) can be subjected to a proprotein convertase substrate assay to determine whether the reagent will inhibit or prevent the activity of a proprotein convertase, thereby preventing the activation of the recombinant protein. The methods of the present disclosure may include performing a proprotein convertase substrate assay. In some embodiments the methods include combining a sample of reagent with a probe that comprises a proprotein convertase recognition site that emits a detectable signal when cleaved at the recognition site and a cognate proprotein convertase, thereby forming a mixture. The mixture may be incubated under conditions that result in cleavage of the probe at the proprotein convertase recognition site. The purpose of the probe is to provide a measureable readout of proprotein convertase cleavage activity. This readout can be used to determine whether a particular reagent prevents proprotein convertase cleavage activity.

### Assessing Proprotein Convertase Activity

Methods for identifying a reagent for use in an assay (e.g., a cell culture based assay) are carried out, in some embodiments, by performing a proprotein convertase substrate assay on a sample of a reagent. In some embodiments, methods for identifying a reagent (e.g., a cell culture medium) for use in the production of a recombinant protein are provided. Certain recombinant proteins (e.g., those containing a proprotein convertase recognition site) can be cleaved by a proprotein convertase, which modulates the activity of the recombinant protein. A “proprotein convertase,” as used herein, refers to an endoprotease that activates another protein. Generally, a proprotein convertase cleaves an inactive (e.g., precursor) protein, thereby producing a biologically active (e.g., mature) form of the protein. The amino acid sequence cleaved by a proprotein convertase is referred to as a proprotein convertase recognition site.

Examples of proprotein convertases for use in accordance with the present disclosure include, without limitation, those listed in Table 1: PCSK1, PCSK2, PCSK3/furin, PCSK4, PCSK5, PCSK6, PCSK7, PCSK8, PCSK9, subtilisin or kexin (SEQ ID NOS: 1-11).

TABLE 1
List of mammalian proprotein convertases
including alternative gene names.

| PCSK Name | Other common names |
|---|---|
| PCSK1 | PC1, PC3, SPC3, NEC1, BMIQ12 |
| PCSK2 | PC2, SPC2, NEC2 |
| PCSK3 | FUR, PACE, SPC1, FURIN |
| PCSK4 | PC4, SPC5 |
| PCSK5 | PC5, PC6, PC6A, SPC6 |
| PCSK6 | PACE4, SPC4 |
| PCSK7 | PC7, PC8, LPC, SPC7 |
| PCSK8 | S1P, SKI-1, MBTPS1 |
| PCSK9 | NARC1, FH3, PC9, LDLCQ1, HCHOLA3 |

In some embodiments, a proprotein convertase comprises (i) a catalytic domain that hydrolyzes a peptide bond of a protein containing a proprotein convertase recognition site, and (ii) a binding pocket that binds to a protein containing a proprotein convertase recognition site.

A proprotein convertase may be obtained from any mammal including, without limitation, humans or rodents (e.g., mice, rats, hamsters). Examples of human proprotein convertases, without limitation, are listed in Table 1.

In some embodiments, a proprotein convertase is homologous to a proprotein convertase selected from the group consisting of: PCSK1, PCSK2, PCSK3/furin, PCSK4, PCSK5, PCSK6, PCSK7, PCSK8, PCSK9, subtilisin, and kexin. For example a proprotein convertase may be at least 70% identical, at least 80% identical, at least 90% identical, at least 95% identical, at least 96% identical, at least 97% identical, at least 98% identical, at least 99% identical, at least 99.5% identical, or at least about 99.9% homologous to PCSK1, PCSK2, PCSK3/furin, PCSK4, PCSK5, PCSK6, PCSK7, PCSK8, PCSK9, subtilisin or kexin (e.g., SEQ ID NOS: 1-11).

Typically, a proprotein convertase cleaves a substrate (e.g., a protein) having a proprotein convertase recognition site. A “proprotein convertase recognition site,” as used herein, is an amino sequence that can be cleaved by a proprotein convertase, as described herein. Methods for predicting proprotein convertase cleavage sites and testing cleavage of the cleavage sites by their cognate proprotein convertase are described in the art. See e.g., Duckert, P., et al., Prediction of proprotein convertase cleavage sites. Protein Engineering Design and Selection 2004. In some embodiments, the proprotein convertase recognition site comprises the amino acid sequence R-X-X-R, where R is arginine and X is any amino acid. In some embodiments, the proprotein convertase recognition site comprises the amino acid sequence R-X-(K/R)-R (SEQ ID NO: 12), where R is arginine, K is lysine and X is any amino acid. In some embodiments, the proprotein convertase recognition site comprises the amino acid sequence R-V-R-R (SEQ ID NO: 13), where R is arginine and V is valine. As used herein, a molecule having a proprotein convertase recognition site is referred to as a “proprotein convertase substrate”.

A proprotein convertase substrate assay, in some embodiments, may be used to identify a reagent for use in the production of a recombinant protein. A “reagent,” as used herein, is a substance or mixture of substances used in a chemical or biological reaction to detect, measure, examine, or produce another substance (e.g., protein or chemical). In some embodiments, a reagent is a cell culture medium. It should be appreciated that, in some embodiments, the cell culture medium may be supplied in the form of a solid material, such as a dry powder (e.g., a crystalline powder). The solid (e.g., dry powder) cell culture medium, in some embodiments, is dissolved or suspended in a liquid solvent (e.g., water, an aqueous salt solution, a buffer solution, or other solvent or any combination of two or more thereof) for use in accordance with the methods described herein. In some embodiments, the cell culture medium is supplied in liquid form, for example as an aqueous solution or suspension. Examples of cell culture medium include, without limitation, Chinese Hamster Ovary (CHO) medium (e.g., OptiCHO™), DMEM, DMEM F12, Ham's Nutrient Mixtures, Medium 199, Minimum Essential Medium Eagle, RPMI Medium, Ames MPF™ Medium, BGJb Medium (Fitton-Jackson Modification), Click's Medium, CMRL-1066 Medium, Fischer's Medium, Glasgow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, and William's Medium E.

It should be appreciated that, in some embodiments, an assay provided herein can be performed on fresh medium or spent medium (e.g., medium that has been depleted of nutrients, dehydrated, or accumulated toxic metabolic products, for example, following cell growth).

In some embodiments, a reagent (e.g., cell culture medium) comprises at least one supplement. For example, a cell culture medium may comprise at least one supplement selected from the group consisting of amino acids, vitamins, antibiotics, antimycotics, cytokines, growth factors, hormones, lipids, lipid carriers, albumins, transport proteins (e.g., transferrin), serum, or serum components. In some embodiments, the reagent is cell-free (i.e., the reagent does not comprise a cell).

In some embodiments, the methods for identifying a reagent for use in the production of a recombinant protein include performing a proprotein convertase substrate assay on a sample of reagent. A “sample” of a reagent, as used herein, refers to a portion of a reagent. A sample, in some embodiments, may have a volume of 1 μL to 10 mL. For example, the sample may have a volume from 1 μL to 10 μL, from 1 μL to 50 μL, from 1 μL to 100 μL, from 1 μL to 500 μL, from 1 μL to 1 mL, from 1 μL to 5 mL, from 1 μL to 8 mL, from 10 μL, to 50 μL, from 10 μL to 100 μL, from 10 μL to 500 μL, from 10 μL to 1 mL, from 10 μL to 5 mL, from 10 μL to 8 mL, from 10 μL to 10 mL, from 50 μL to 100 μL, from 50 μL to 500 μL, from 50 μL to 1 mL, from 50 μL to 5 mL, from 50 μL to 8 mL, from 50 μL to 10 mL, from 100 μL to 500 μL, from 100 μL to 1 mL, from 100 μL to 5 mL, from 100 μL to 8 mL, from 100 μL to 10 mL, from 500 μL to 1 mL, from 500 μL to 5 mL, from 500 μL to 8 mL, from 500 μL to 10 mL, from 1 mL to 5 mL, from 1 mL to 8 mL, from 1 mL to 10 mL, from 5 mL to 8 mL, from 5 mL to 10 mL, or from 8 mL to 10 mL obtained from a container.

In some embodiments, a sample is provided in a dry or solid form (e.g., as a dry powder), for example in a defined amount, that is added to a liquid reaction mixture in order to evaluate the amount of inhibitory activity that is contained in the sample.

In some embodiments, the suitability of one reagent (e.g., cell culture medium) is compared to the suitability of another reagent (e.g., suitability for use in producing a recombinant protein). Thus, in some embodiments, where more than one sample is tested, the samples may be obtained from reagents that have the same batch and/or lot number, or, alternatively, the samples may be obtained from reagents that have different batch and/or lot numbers.

The methods of the present disclosure can be used to identify a reagent (e.g., a suitable reagent) for use in the production of a protein. In some embodiments, the protein is a recombinant protein. A “recombinant protein,” as used herein, is a non-naturally occurring polypeptide. Typically, recombinant proteins are produced using DNA molecules that are formed by genetic recombination to create sequences that would not otherwise be found in nature. For example, the recombinant proteins described herein may be produced via recombinant protein expression and purification. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference. In some embodiments, the recombinant proteins described herein comprise a proprotein convertase recognition site. Examples of recombinant proteins that may be used in accordance with the disclosure include, but are not limited to, Factor VIII, unmodified rBDD FIX (ReFacto/Xyntha), and FIXFc.

Methods for identifying a reagent, e.g., a suitable reagent, for use in the production of a recombinant protein, in some embodiments, includes performing a proprotein convertase substrate assay. A proprotein convertase substrate assay, in some embodiments, is used to determine whether a reagent used in the production of a recombinant protein inhibits the processing of the recombinant protein, for example, by inhibiting a proprotein convertase that cleaves and activates the recombinant protein. A “proprotein convertase substrate assay,” as used herein, is an analytic procedure for assessing the ability of a proprotein convertase to cleave a molecule (e.g., a molecule having a proprotein convertase recognition site) under experimental or physiological conditions. In some embodiments, the activity of a proprotein convertase is assessed by detecting a signal emitted from a proprotein convertase substrate (e.g., a probe) when it is cleaved. In some embodiments, the activity of a proprotein convertase is assessed by contacting the proprotein convertase with a proprotein convertase substrate that emits a detectable signal when cleaved at the recognition site. For example a proprotein convertase substrate may comprise a fluorescent probe that is detectable upon cleavage of the proprotein convertase substrate by a proprotein convertase. The amount of fluorescent signal detected in some embodiments is used to assess the activity of a proprotein convertase in the presence or absence of a sample of a reagent.

In some embodiments, performing a proprotein convertase substrate assay includes combining a sample of reagent with a probe that comprises a proprotein convertase recognition site that emits a detectable signal when cleaved at the recognition site. As used herein, a “probe that comprises a proprotein convertase recognition site” or “probe”, refers to a synthetic molecule that has a proprotein convertase recognition site. The term “synthetic” as used herein means synthesized chemically, synthesized recombinantly, or not in an amount or purity found in nature. In some embodiments, “synthetic” refers to a molecule that does not occur in nature. A “synthetic probe” refers to a molecule comprising a proprotein convertase recognition site that is synthesized chemically, synthesized recombinantly or by other means. The probes, e.g., synthetic probes, of the present disclosure include those that are chemically modified, or otherwise modified, but can be cleaved by a proprotein convertase. It should be understood that while a synthetic probe as a whole is not naturally-occurring, it may include amino acid sequences that occur in nature.

The probe may comprise an amino acid sequence of any suitable length for use in accordance with the methods, described herein. In some embodiments, the probe comprises an amino acid sequence that is at least four amino acids (aa) in length. In some embodiments, the probe comprises an amino acid sequence ranging from 4 aa to 1,000 aa in length. In some embodiments, the probe comprises an amino acid sequence ranging in length from 4 aa to 10 aa, from 4 aa to 20 aa, from 4 aa to 50 aa, from 4 aa to 100 aa, from 4 aa to 200 aa, from 4 aa to 400 aa, from 4 aa to 600 aa, from 4 aa to 800 aa, from 10 aa to 20 aa, from 10 aa to 50 aa, from 10 aa to 100 aa, from 10 aa to 200 aa, from 10 aa to 400 aa, from 10 aa to 600 aa, from 10 aa to 800 aa, from 10 aa to 1000 aa, from 20 aa to 50 aa, from 20 aa to 100 aa, from 20 aa to 200 aa, from 20 aa to 400 aa, from 20 aa to 600 aa, from 20 aa to 800 aa, from 20 aa to 1000 aa, from 50 aa to 100 aa, from 50 aa to 200 aa, from 50 aa to 400 aa, from 50 aa to 600 aa, from 50 aa to 800 aa, from 50 aa to 1000 aa, from 100 aa to 200 aa, from 100 aa to 400 aa, from 100 aa to 600 aa, from 100 aa to 800 aa, from 100 aa to 1000 aa, from 200 aa to 400 aa, from 200 aa to 600 aa, from 200 aa to 800 aa, from 200 aa to 1000 aa, from 400 aa to 600 aa, from 400 aa to 800 aa, from 400 aa to 1000 aa, from 600 aa to 800 aa, from 600 aa to 1000 aa, or from 800 aa to 100 aa. It should be appreciated that the probe may comprise an amino acid sequence of any length that can be cleaved by a proprotein convertase to emit a detectable signal.

The probes of the present disclosure are designed, in some embodiments, to emit a detectable signal when cleaved at the recognition site. As used herein, “cleaved at the recognition site” means that at least one peptide bond within a proprotein recognition site of a probe is cut, thereby partitioning the probe into at least two molecules. Cleavage of the probe at the recognition site, in some embodiments emits a detectable signal. A “detectable signal”, as used herein, refers to any product resulting from the cleavage of the probe that can be measured or detected, for example, by using a signal detection assay. In some embodiments the detectable signal is a fluorescent signal, the size of the probe and/or any fragments thereof (e.g., a signal reflecting the size of a probe fragment), or a chemiluminescent signal.

In some embodiments, the detectable signal is a fluorescent signal. A “fluorescent signal” as used herein refers to the emission of light by a substance (e.g., a fluorophore) that has absorbed light or other electromagnetic radiation. Typically, the emitted light has a longer wavelength, and therefore lower energy than the absorbed radiation. It should be appreciated that a fluorophore may have different fluorescent properties depending on the environment (e.g., pH), or whether the fluorophore is bound to another molecule. For example the fluorophore may absorb and emit energy (e.g., light energy) differently depending on whether it is bound to a molecule or whether it is free from the molecule. Accordingly, a fluorescent signal may be used, according to the methods described herein, to determine whether a fluorophore is bound to a molecule (e.g., a probe). In some embodiments, the fluorescent signal is light emitted from a fluorophore bound to a probe that has absorbed light. In some embodiments, the fluorescent signal is light emitted form a fluorophore that has been released from a probe (e.g., by cleavage of the probe by a proprotein convertase) and that has absorbed light. It should be appreciated that the absorption and emission properties will typically depend on the fluorophore being used, the environment in which the fluorophore is detected and the molecule to which the fluorophore may be bound.

In some embodiments the detectable signal is a fluorescent signal having a wavelength ranging from 150 nanometers (nm) to 1000 nm. In some embodiments, the detectable signal is a fluorescent signal having a wavelength ranging from 150 nm to 200 nm, from 150 nm to 300 nm, from 150 nm to 350 nm, from 150 nm to 400 nm, from 150 nm to 450 nm, from 150 nm to 500 nm, from 150 nm to 600 nm, from 150 nm to 700 nm, from 150 nm to 800 nm, from 150 nm to 900 nm, from 200 nm to 300 nm, from 200 nm to 350 nm, from 200 nm to 400 nm, from 200 nm to 450 nm, from 200 nm to 500 nm, from 200 nm to 600 nm, from 200 nm to 700 nm, from 200 nm to 800 nm, from 200 nm to 900 nm, from 300 nm to 350 nm, from 300 nm to 400 nm, from 300 nm to 450 nm, from 300 nm to 500 nm, from 300 nm to 600 nm, from 300 nm to 700 nm, from 300 nm to 800 nm, from 300 nm to 900 nm, from 350 nm to 400 nm, from 350 nm to 450 nm, from 350 nm to 500 nm, from 350 nm to 600 nm, from 350 nm to 700 nm, from 350 nm to 800 nm, from 350 nm to 900 nm, from 400 nm to 450 nm, from 400 nm to 500 nm, from 400 nm to 600 nm, from 400 nm to 700 nm, from 400 nm to 800 nm, from 400 nm to 900 nm, from 450 nm to 500 nm, from 450 nm to 600 nm, from 450 nm to 700 nm, from 450 nm to 800 nm, from 450 nm to 900 nm, from 500 nm to 600 nm, from 500 nm to 700 nm, from 500 nm to 800 nm, from 500 nm to 900 nm, from 600 nm to 700 nm, from 600 nm to 800 nm, from 600 nm to 900 nm, from 700 nm to 800 nm, from 700 nm to 900 nm, from 800 nm to 900 nm. In some embodiments the detectable signal is a fluorescent signal emitted from 7-Amino-4-methylcoumarin (AMC). In some embodiments, AMC is excited by light having a wavelength ranging from 360 nm to 400 nm. In some embodiments the AMC emits light having a wavelength ranging from 420 nanometers (nm) to 420 nm.

A “fluorophore,” as used herein, is any chemical compound that can emit light upon light excitation. In some embodiments, the fluorophore is a molecule that has different fluorescent properties when it is bound to a molecule (e.g., a probe comprising an amino acid) versus when it is released from the molecule. As one non-limiting example, the fluorophore 7-Amino-4-methylcoumarin (AMC) fluoresces very weakly when bound to an amino acid sequence (e.g., a proprotein convertase recognition site) but when AMC is released, for example by a proprotein convertase, it fluoresces strongly. Fluorophores that may be used in accordance with the disclosure include, without limitation, fluorescent proteins (e.g., green fluorescent protein, red fluorescent protein and yellow fluorescent protein), non-protein organic fluorophores (e.g., xanthene derivatives, cyanine derivatives, squaraine derivatives, naphthalene derivatives, coumarin derivatives, oxadiazole derivatives, anthracene derivatives, pyrene derivatives, oxazine derivatives, acridine derivatives, arylmethine derivatives, and tetrapyrrole derivatives), and other commercially available fluorophores including CF™ dye (Biotium), DRAQ™ and CyTRAK™ probes (BioStatus), BODIPY® (Invitrogen), Alexa Fluor® (Invitrogen), DyLight® Fluor (Thermo Scientific, Pierce), Atto and Tracy (Sigma Aldrich), FluoProbes® (Interchim), Abberior Dyes (Abberior), DY and MegaStokes Dyes (Dyomics), Sulfo Cy dyes (Cyan Dye), HiLyte Fluor™ (AnaSpec), Seta, SeTau and Square Dyes (SETA BioMedicals), Quasar® and Cal Fluor® dyes (Biosearch Technologies), SureLight Dyes (APC, RPEPerCP, Phycobilisomes) (Columbia Biosciences), APC, APCXL, RPE, BPE (Phyco-Biotech). In some embodiments, the fluorophore used in accordance with the disclosure is 7-Amino-4-methylcoumarin (AMC).

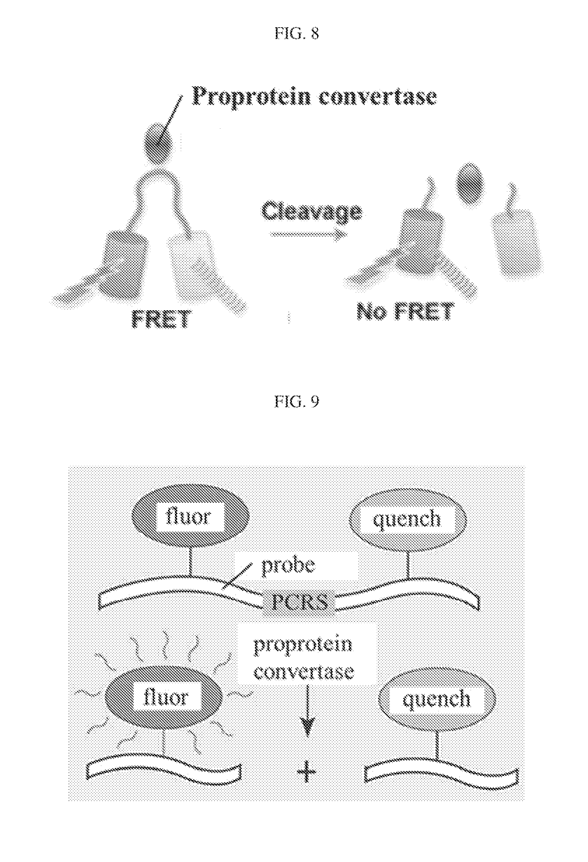

The probes of the present disclosure, in some embodiments, comprise a fluorophore. For example, the probe may have a single fluorophore that emits a detectable fluorescent signal upon cleavage of the probe (e.g., by a proprotein convertase). In some embodiments, the probe comprises two or more fluorophores. In some embodiments, the probe comprises 2, 3, 4, 5, 6, 7, 8, 9, 10, 11, 12, 13, 14, 15, 16, 17, 18, 19, 20 or more fluorophores. In some embodiments, 2 or more fluorophores are configured on the probe to produce at least one fluorescence resonance energy transfer (FRET) signal. FRET is a mechanism of energy transfer between two light-sensitive molecules (e.g., fluorophores). For example a donor fluorophore may transfer energy (e.g., light energy) to an acceptor fluorophore through nonradiative dipole-dipole coupling. The efficiency of this energy transfer is inversely proportional to the distance between the donor and acceptor fluorophore. Accordingly, the FRET signal can be used to determine whether the two fluorophores are within a certain distance of each other. As one example the probe may have a first fluorophore that is amino-terminal to the proprotein recognition site of the probe and a second fluorophore that is carboxy-terminal to the proprotein recognition site of the probe. In some embodiments, the first fluorophore and the second fluorophore are configured to produce a FRET signal when either the first or the second fluorophore is excited (e.g., by a light of a specific wavelength). In some embodiments, cleavage of the probe at a proprotein convertase recognition site separates the fluorophores, thereby decreasing or eliminating the FRET signal. A non-limiting example of a probe being cleaved by a proprotein convertase to prevent a FRET signal is shown in FIG. 8. In some embodiments, a FRET signal is detected when the probe is not cleaved and a FRET signal is absent when the probe is cleaved (e.g., by a proprotein convertase).

In some embodiments, the probes of the present disclosure comprise a quenching molecule. A “quenching molecule” as used herein is a molecule or compound that decreases the fluorescence intensity of a substance (e.g., a fluorophore). A quenching molecule may absorb all, or a portion of the light energy emitted by a fluorophore. In some embodiments the quenching molecule is a dark quenching molecule. A dark quenching molecule is a molecule that absorbs energy from a fluorophore and dissipates the energy as heat. In some embodiments, the probe comprises two or more quenching molecules. In some embodiments, the probe comprises 2, 3, 4, 5, 6, 7, 8, 9, 10, 11, 12, 13, 14, 15, 16, 17, 18, 19, 20 or more quenching molecules. In some embodiments, a quenching molecule is configured on the probe to absorb a fluorescent signal from a fluorophore on the probe. As one example the probe may have a quenching molecule that is amino-terminal to the proprotein recognition site of the probe and a fluorophore that is carboxy-terminal to the proprotein recognition site of the probe. As another example the probe may have a quenching molecule that is carboxy-terminal to the proprotein recognition site of the probe and a fluorophore that is amino-terminal to the proprotein recognition site of the probe. In some embodiments, a quenching molecule and a fluorophore are configured on the probe such that when the fluorophore is excited (e.g., by light at a specific wavelength) the quenching molecule absorbs the light emitted from the fluorophore, thereby preventing or inhibiting the detectable signal from the fluorophore. In some embodiments, cleavage of the probe at a proprotein convertase recognition site separates the fluorophore from the quencher, thereby allowing the fluorophore to produce a detectable signal. A non-limiting example of a probe with a quencher and a fluorophore being cleaved by a proprotein convertase is shown in FIG. 9. In some embodiments, a fluorescence signal is detected when the probe is cleaved (e.g., by a proprotein convertase) and a fluorescence signal is not detected when the probe is not cleaved. Exemplary quenching molecules within the scope of the disclosure include, but are not limited to, dimethylaminoazobenzenesulfonic acid (Dabsyl), Black hole quenchers, Qx1 quenchers, iowa black FQ, Iowa black RQ and IRCye QC-1.

In some embodiments, the detectable signal is a size of a probe or fragment of the probe. For example, the detectable signal may be a fragment of a probe that has been cut by a proprotein convertase, which may be detected using an assay capable of distinguishing the size of a molecule, such as sodium dodecyl sulphate-polyacrylamide gel electrophoresis (SDS-PAGE) and Western Blotting. Other suitable methods for detecting the size of a probe include, without limitation, size exclusion chromatography and mass spectroscopy.

The probes of the present disclosure, in some embodiments, comprise a “protecting group.” A “protecting group”, as referred to herein, is a molecule other than an amino acid that can be added to the amino-terminus or carboxy-terminus of an amino acid molecule. Typically, protecting groups are introduced into amino acid molecules by chemical modification of a functional group (e.g., an amine group or a carboxylic acid group) to protect them from reagents in the environment or reagents used during organic synthesis (e.g., to prevent polymerization). In some embodiments, the probe comprises an amine protecting group. An amine protecting group may comprise, without limitation, a Carbobenzyloxy (Cbz) group, a p-Methoxybenzyl carbonyl (Moz or MeOZ) group, a tert-Butyloxycarbonyl (BOC) group, a 9-Fluorenylmethyloxycarbonyl (FMOC) group, an Acetyl (Ac) group, a Benzoyl (Bz) group, a Benzyl (Bn) group, a p-Methoxybenzyl (PMB) group, a 3,4-Dimethoxybenzyl (DMPM) group, a p-methoxyphenyl (PMP) group, a Tosyl (Ts) group, or other sulfonamide (Nosyl & Nps) groups. In some embodiments, a probe comprises a carboxylic acid protecting group. A carboxylic acid protecting may comprise, without limitation, Methyl esters, Benzyl esters, tert-Butyl esters, Esters of 2,6-disubstituted phenols (e.g., 2,6-dimethylphenol, 2,6-diisopropylphenol, 2,6-di-tert-butylphenol), Silyl esters, Orthoesters, or Oxazoline. In some embodiments, the probe comprises a tert-butyloxycarbonyl (t-Boc) protecting group. In some embodiments, the probe comprises a 9-fluorenylmethyloxycarbonyl (Fmoc) protecting group.

In some embodiments, the probes disclosed herein have a protecting group, a fluorophore and a linker that comprises a proprotein convertase recognition site. In some embodiments, the probe has a protecting group that is linked to a fluorophore via a linker comprising a proprotein convertase recognition site. The linker, as described herein, refers to any molecule comprising a proprotein convertase recognition site. The linker has an amino-terminal end that is located amino-terminally to the proprotein convertase recognition site and a carboxy-terminal end that is located carboxy-terminally to the proprotein convertase recognition site. The linker may comprise any molecule (e.g., an amino acid, an amino acid sequence, a fluorophore, a protecting group, or another molecule capable of binding a fluorophore or protecting group) at the amino-terminus and/or the carboxy-terminus of the proprotein convertase recognition site. In some embodiments, the linker consists of a proprotein recognition site. In some embodiments, the protecting group is located at the amino-terminal end of the linker. For example, the protecting group, without limitation, may be bound to the amino-terminus of a proprotein recognition site, or to another portion of the linker that is amino-terminal to the proprotein recognition site. In some embodiments, the protecting group is located at the carboxy-terminal end of the linker. For example, the protecting group, without limitation, may be bound to the carboxy-terminus of a proprotein convertase recognition site, or to another portion of the linker that is carboxy-terminal to the proprotein recognition site. In some embodiments, the fluorophore is located at the amino-terminal end of the linker. For example, the fluorophore, without limitation, may be bound to the amino-terminus of a proprotein recognition site, or to another portion of the linker that is amino-terminal to the proprotein recognition site. In some embodiments, the fluorophore is located at the carboxy-terminal end of the linker. For example, the fluorophore, without limitation, may be bound to the carboxy-terminus of a proprotein convertase recognition site, or to another portion of the linker that is carboxy-terminal to the proprotein recognition site. In some embodiments, the probe comprises a fluorophore that is located at the amino-terminal end of the linker and a protecting group that is located at the carboxy-terminal end of the linker. In some embodiments, the probe comprises a fluorophore that is located at the amino-terminal end of a proprotein convertase recognition site and a protecting group that is located at the carboxy-terminal end of the proprotein recognition site. In some embodiments, the probe comprises a protecting group that is located at the amino-terminal end of the linker and a fluorophore that is located at the carboxy-terminal end of the linker. In some embodiments, the probe comprises a protecting group that is located at the amino-terminal end of a proprotein convertase recognition site and a fluorophore that is located at the carboxy-terminal end of the proprotein recognition site. In some embodiments, the probe comprises a t-Boc protecting group linked to AMC via a linker. In some embodiments, the probe comprises a t-Boc protecting group bound at the amino-terminal end of the amino acid sequence RVRR (SEQ ID NO: 13), where R is arginine and V is valine, and AMC bound at the carboxy-terminal end of the amino acid sequence RVRR (SEQ ID NO: 13) (e.g., t-Boc/RVRR/AMC). In some embodiments, the probe consists of (t-Boc/RVRR/AMC).

In some embodiments, performing a proprotein convertase substrate assay comprises combining a sample of a reagent with a probe and a cognate proprotein convertase, thereby forming a mixture. A “cognate proprotein convertase,” as used herein, refers to a proprotein convertase that is capable of cleaving a specific proprotein convertase recognition site. For example, the proprotein convertase PCSK3/furin is capable of cleaving the proprotein convertase recognition site RVRR (SEQ ID NO: 13). Accordingly, PCSK3/furin is a cognate proprotein convertase to the proprotein convertase recognition site RVRR (SEQ ID NO: 13). A “mixture,” as used herein, refers to the combination of at least two substances. In some embodiments the mixture is a combination of at least 3, at least 4, at least 5 at least 10 at least 20 at least 50, or at least 100 substances. In some embodiments, the mixture comprises a sample of a reagent, a probe, and a cognate proprotein convertase capable of cleaving the probe at the proprotein convertase recognition site. In some embodiments, the mixture further comprises a buffer (e.g., HEPES, TRIS, MES, and MOPS), a detergent (e.g., TRITON® X-100, TWEEN® 20, CHAPS, and Sodium Dodecyl Sulfate), a reducing agent, (e.g., 2-mercaptoethanol, dithiothreitol, and tris(2-carboxyethyl)phosphine), a salt (e.g., NaCl, CaCl₂, MgSO₄, and ZnCl₂), an acid (e.g., HCl, and H₂SO₄), a base (e.g., NaOH), or any other reagent that may be used in a proprotein convertase substrate assay, or any combination of two or more thereof. It should be appreciated that the substances of the mixture, described herein, are exemplary and that additional substances are also within the scope of the disclosure.

The proprotein convertase substrate assay, in some embodiments, comprises incubating the mixture under conditions that result in the cleavage of the probe at the proprotein convertase recognition site. As used herein, “conditions that result in the cleavage of the probe at the proprotein recognition site” or “cleavage conditions” refer to experimental conditions under which a proprotein convertase substrate is capable of being cleaved by a cognate proprotein convertase. It should be appreciated that, in general, the cleavage conditions of the present disclosure will differ depending on the proprotein convertase being used, the proprotein convertase substrate being cleaved and/or the fluorophore being detected. Accordingly, the cleavage conditions may be modified to achieve a desired result (e.g., cleavage of a probe by a proprotein convertase). In some embodiments, the cleavage conditions that may be modified in accordance with the disclosure include but are not limited to the type and concentration of buffer, the type and concentration of salt, the type and concentration of detergent, the type and concentration of reducing agent, the pH, the temperature, the volume of the proprotein convertase substrate assay, and the duration of the assay (e.g., incubation time). In some embodiments, the pH is adjusted to modulate the cleavage conditions, which can affect proprotein convertase activity (see, e.g., FIG. 9). In some embodiments, the cleavage conditions include pH ranges from 6.8 to 7.4 or from 7.0 to 7.2. However, it should be appreciated that other proprotein convertases may respond differently to changes in pH, which may be modified for use with any specific proprotein convertase. Methods for performing a proprotein convertase substrate assay are known in the art and have been described previously. Examples include, without limitation, Molloy, S. S., et al., “Human Furin Is a Calcium-dependent Serine Endoprotease That Recognizes the Sequence Arg-X-X-Arg and Efficiently Cleaves Anthrax Toxin Protective Antigen” J. Biol. Chem., Vol. 267, No. 23, August 15, pp. 16396-16402, (1992).

In some embodiments, a proprotein convertase substrate assay further comprises performing a signal detection assay on the mixture. A “signal detection assay,” as described herein is an analytic procedure that can be used to detect a signal (e.g., a fluorescent signal) in a mixture. In some embodiments, the signal detection assay is a spectrofluorometric assay. A spectrofluorometric assay, as used herein, is an analytic method used to measure the fluorescent properties of a substance (e.g., a fluorophore) in a mixture. The spectrofluorometric assay may be performed using any suitable instrument capable of detecting the fluorescent properties of a fluorescent substance (e.g., a fluorophore) in a mixture. In some embodiments, a spectrofluorometric assay is performed using a spectrofluorometer, which is an instrument that takes advantage of fluorescent properties of compounds (e.g., fluorophores) in order to provide information regarding their concentration and chemical environment in a mixture. Typically, a certain excitation wavelength is selected, and the emission is observed either at a single wavelength, or a scan is performed to record the intensity versus wavelength, also called an emission spectra. In some embodiments, a spectrofluorometric assay comprises subjecting a mixture (e.g., a mixture from a proprotein convertase substrate detection assay) to light energy at a first wavelength (e.g., an excitation wavelength) and detecting light energy from the mixture at a second wavelength (e.g., an emission wavelength). In some embodiments the amount of light energy emitted by a fluorophore (e.g., an excited fluorophore) is used to determine the amount or proportion of the probe in a proprotein convertase substrate assay that has been cleaved at the recognition site. In some embodiments, the amount of fluorescence detected in the mixture is proportional to the amount of activity of the cognate proprotein convertase in the sample of the reagent.

In some embodiments, the signal detection assay is an assay capable of detecting the size of a substance (e.g., a probe or fragment of a probe) in a mixture. For example, the signal detection assay may comprise separating substances (e.g., probes and probe fragments) by size using SDS-PAGE and detecting a probe or probe fragment (e.g., following cleavage by a proprotein convertase) by Western Blotting. In some embodiments, the signal detection assay comprises size exclusion chromatography. Size Exclusion Chromatography (SEC) is a separation technique based on the molecular size of components (e.g., compounds in a mixture). Separation by SEC is typically achieved by the differential exclusion of sample molecules from the pores of a packing material as they pass through a bed of porous particles. It should be appreciated that any detection assay capable of detecting the size of a probe may be used to determine the amount or proportion of a probe in a proprotein convertase substrate assay that has been cleaved at the recognition site. In some embodiments the amount of a probe cleavage product detected (e.g., a probe fragment resulting from cleaving a probe with a proprotein convertase) is used to determine the amount or proportion of the probe in a proprotein convertase substrate assay that has been cleaved at the recognition site. In some embodiments, the amount of a probe cleavage product detected in the mixture is proportional to the amount of activity of the cognate proprotein convertase in the sample of the reagent.

In some embodiments, methods for identifying a reagent for use in the production of a recombinant protein further include comparing the signal detected in a first mixture to a signal detected in a second mixture. Any of the methods described herein may further include comparing a signal detected in a first mixture (e.g., a mixture from a proprotein convertase substrate assay) containing a sample of a first reagent, to a signal detected in a second mixture, containing a sample of a second reagent. In some embodiments, the first reagent of the first mixture is a control reagent (e.g., PBS). A control reagent is a reagent that does not to inhibit the activity of a proprotein convertase. The purpose of the control reagent in some embodiments is measure the activity of a proprotein convertase under certain cleavage conditions in the absence of a proprotein convertase inhibitor. The activity of a proprotein convertase in the presence of a control reagent can be compared to the activity of a proprotein convertase in the presence of a reagent (e.g., a second reagent) in order to determine whether the reagent inhibits the activity of the proprotein convertase. In some embodiments, the methods further comprise selecting the second reagent. In some embodiments the second reagent is selected if the second reagent inhibits a proprotein convertase by no more than 60%, no more than 50%, no more than 40%, no more than 30%, no more than 20%, no more than 10%, no more than 5%, no more than 2%, or no more than 1% as compared to the activity of a proprotein convertase in the presence of a control reagent (e.g., PBS).

In some embodiments, the first sample of reagent and the second sample reagent are of the same type and are obtained from separate lots. In some embodiments a signal detection assay is performed on the first mixture and the second mixture. In some embodiments, the methods further include detecting a signal in the mixtures by performing a signal detection assay. The amount of signal detected in the first and second mixtures, in some embodiments, is proportional to the amount of activity of a proprotein convertase in the first and second sample of reagent. In some embodiments, the methods further comprise selecting the first sample of reagent used in the first mixture, or selecting the second sample of reagent used in the second mixture based on the amount of signal detected in each of the mixtures.

Aspects of the disclosure relate to a method of identifying cell culture medium suitable for producing recombinant Factor VIII, which is a blood-clotting protein that is sometimes referred to as anti-hemophilic factor (AHF). Cell culture medium may contain inhibitors of a proprotein convertase such as PCSK3/furin. For example, components of cell culture medium may degrade over time, yielding degradation products that interfere or inhibit the activity of PCSK3/furin. Determining whether such inhibitors are present in the medium would be useful for selecting an appropriate medium for producing a recombinant protein that requires processing (e.g., cleavage) by a proprotein convertase such as PCSK3/furin to yield an active recombinant protein product. If, for example, a cell culture medium containing degradation products that inhibit PCSK3/furin activity is used for producing recombinant protein Factor VIII, there may be a significant decrease in the amount of active “processed” Factor VIII because the non-processed isoform (NPI) of Factor III is not cleaved and activated by PCSK3/furin. Accordingly, methods for determining whether the a cell culture medium is suitable for producing “active” processed recombinant Factor VIII are provided. In some embodiments, the method includes performing a PCSK3/furin substrate assay on a sample of cell culture medium. In some embodiments, the methods of performing a PCSK3/furin substrate assay include combining the sample of cell culture medium with a probe that contains a PCSK3/furin recognition site and emits a detectable signal when cleaved at the recognition site, and PCSK3/furin, thereby forming a mixture. The mixture, in some embodiments, is incubated under conditions that result in cleavage of the probe at the PCSK3/furin recognition site. Cleavage of the probe at the PCSK3/furin recognition site may be detected in the mixture using a signal detection assay, for example a fluorescent based detection assay.

In some embodiments, methods of identifying cell culture medium suitable for production of recombinant Factor VIII include detecting a signal in the mixture as a result of performing the signal detection assay, where the amount of signal detected in the mixture is proportional to the amount of activity (e.g., cleavage activity of recombinant Factor VIII) of the PCSK3/furin in a sample of the reagent. In some embodiments, the detectable signal is fluorescent signal. For example, a probe having a PCSK3/furin recognition site and a fluorescent moiety is cleaved by PCSK3/furin, thereby releasing the fluorescent moiety, which may be detected in the reaction mixture by any suitable means.

In some embodiments, the a PCSK3/furin recognition site includes the sequence: R-V-R-R (SEQ ID NO: 13), wherein R is arginine, and V is valine. However, it should be appreciated that the PCSK3/furin recognition site can be any amino acid sequence that is capable of being cleaved by PCSK3/furin.

In some embodiments, methods for identifying cell culture medium suitable for producing recombinant Factor VIII are used to identify suitable cell culture medium that is cell-free or protein-free.

In some embodiments, the methods for identifying cell culture medium suitable for producing recombinant Factor VIII include a probe that has a protecting group linked to a fluorophore via a linker comprising the PCSK3/furin recognition site. The protecting group may be suitable protecting group, such as an amine protecting group. For example, the amine protecting group, in some embodiments is a tert-butyloxycarbonyl (t-Boc) protecting group, or a 9-fluorenylmethyloxycarbonyl (Fmoc) protecting group. It should be appreciated, however, that any suitable protecting group may be used and falls within the scope of the present disclosure.

The fluorophore may be any suitable fluorophore capable of being detected in a PCSK3/furin substrate assay. For example, the fluorophore may be 7-Amino-4-methylcoumarin (AMC).

In some embodiments, the probe comprises a t-Boc protecting group linked to AMC via a linker that comprises the following amino acid sequence: R-V-R-R (SEQ ID NO: 13), wherein R is arginine, and V is valine.

Any of the methods described herein may further include comparing a signal detected in a first mixture (e.g., a mixture from a PCSK3/furin substrate assay) containing a sample of a first reagent, to a signal detected in a second mixture, containing a sample of a second reagent. In some embodiments, the methods may include combining the first and second reagent with a probe having a PCSK3/furin recognition site which emits a detectable signal when cleaved at the recognition site, and PCSK3/furin, thereby producing a first and second mixture and incubating the mixtures under conditions that result in cleavage of the probe in the mixtures. In some embodiments, the first sample of reagent and the second sample reagent are of the same type and are obtained from separate lots. In some embodiments a signal detection assay is performed on the first mixture and the second mixture. In some embodiments, the methods further include detecting a signal in the mixtures as a result of performing the signal detection assay. The amount of signal detected in the first and second mixtures, in some embodiments, is proportional to the amount of activity of PCSK3/furin in the first and second reagents. In some embodiments, the methods further comprise selecting the first reagent used in the first mixture, or selecting the second reagent used in the second mixture based on the amount of signal detected in each of the mixtures.

It should be appreciated that one or more reagents may be provided in suitable containers or vessels (e.g., vials, wells, tubes, or other vessel or container). In some embodiments, assays may be performed in suitable reaction vessels or containers (e.g., vials, wells, tubes, or other vessel or container). In some embodiments, reagents may be provided in and/or assays may be performed in multi-well plates or other suitable multi-sample arrays. In some embodiments, one or more reagents (for example, a probe) may be fixed (e.g., covalently or non-covalently bound) to a surface of a reaction vessel or container. However, in some embodiments the reagents are not fixed to a surface and are freely soluble in the reaction mixture.

Aspects of the invention are further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove.

### EXAMPLES

### Example 1

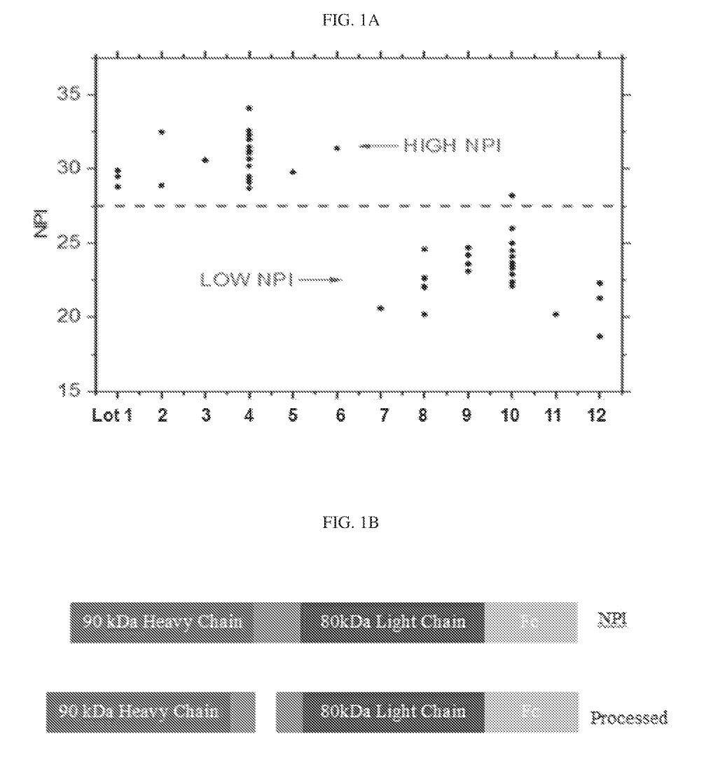

FVIII has exhibited variability in non-processed isoform (NPI) formation. Experimental data indicates that certain lots of OptiCHO™ medium (FIGS. 1A-1B) may explain the variability observed in NPI processing, and NPI variability has been linked to Furin inhibition. See e.g., Bass et al. and Thomas et al., which describe sequential cleavage and degradation of misfolded insulin receptors and various features of furin, including structural and enzymatic properties, trafficking, intracellular localization, substrates and roles in vivo (Bass J., et al., PNAS, 2000 vol. 97 no. 22, p. 11905-11909; Thomas et al., Nat. Rev Mol. Cell Biol., 2002 October; 3(10):753-66.).

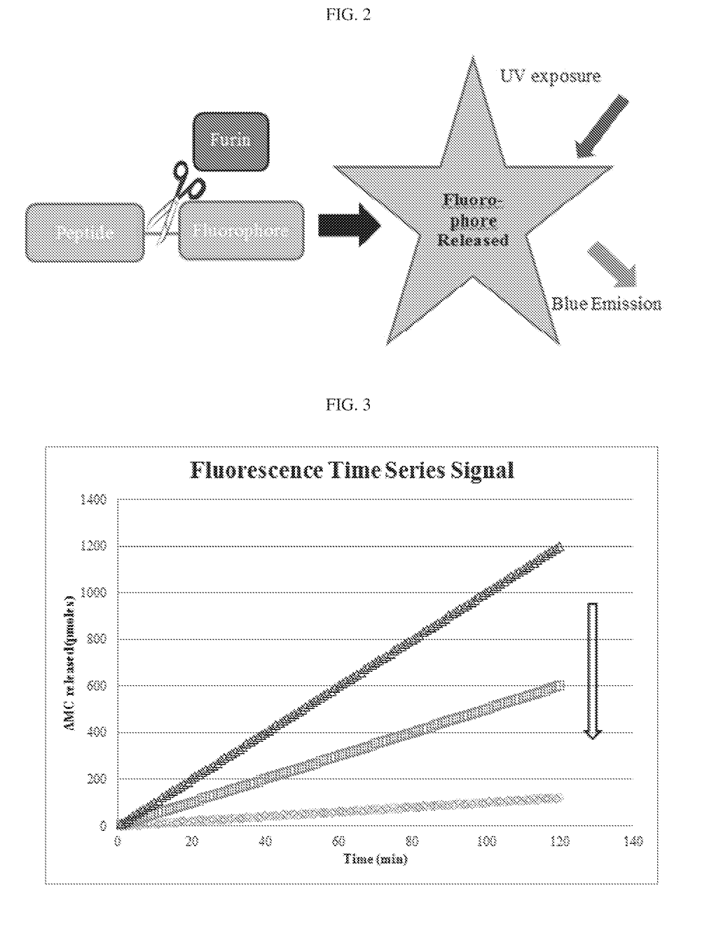

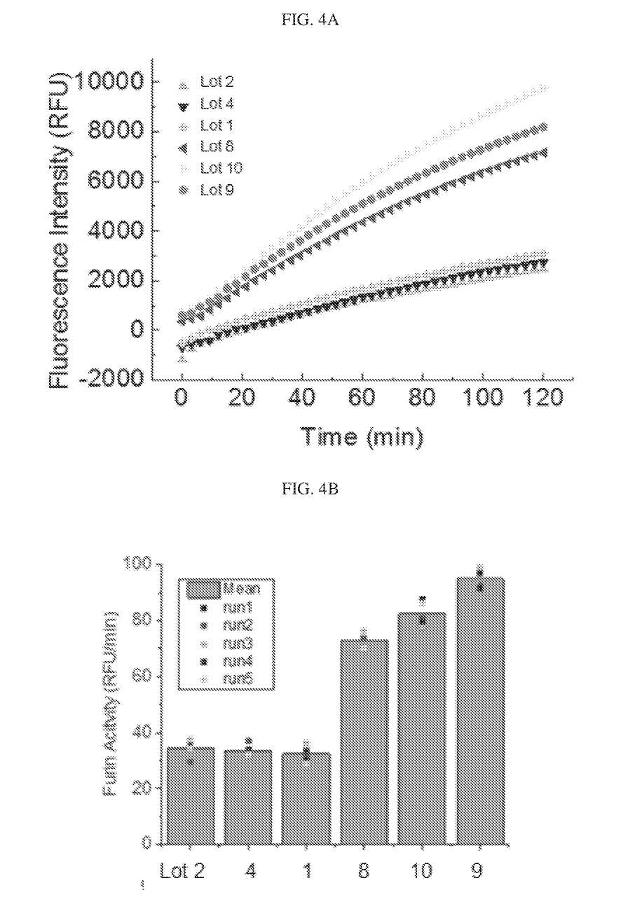

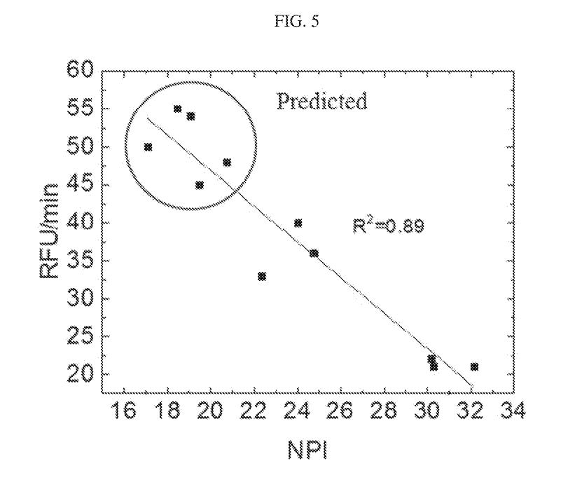

A fluorometric assay was developed to demonstrate furin inhibition. The fluorogenic substrates BOC-RVRR-AMC were labeled with the fluoro-chrome 7 amino-4-methyl coumarin (AMC). Free AMC emits a green-blue fluorescence at 460 nm that can be detected by exposure to UV light at 360 nm. The AMC is released from the BOC-RVRR-AMC substrates upon cleavage by Furin enzymes. The amount of fluorescence produced upon cleavage is proportional to the amount of furin activity present in the sample (FIG. 2). Bourne et al., “Development and characterisation of an assay for furin activity.” J. Immunol Methods, (2011). Inhibition of Furin lead to a decrease in the AMC activity (FIG. 3). In FIGS. 4A-4B, graphs depict furin activity correlating with NPI. In FIG. 5 further data is found correlating furin activity with NPI.

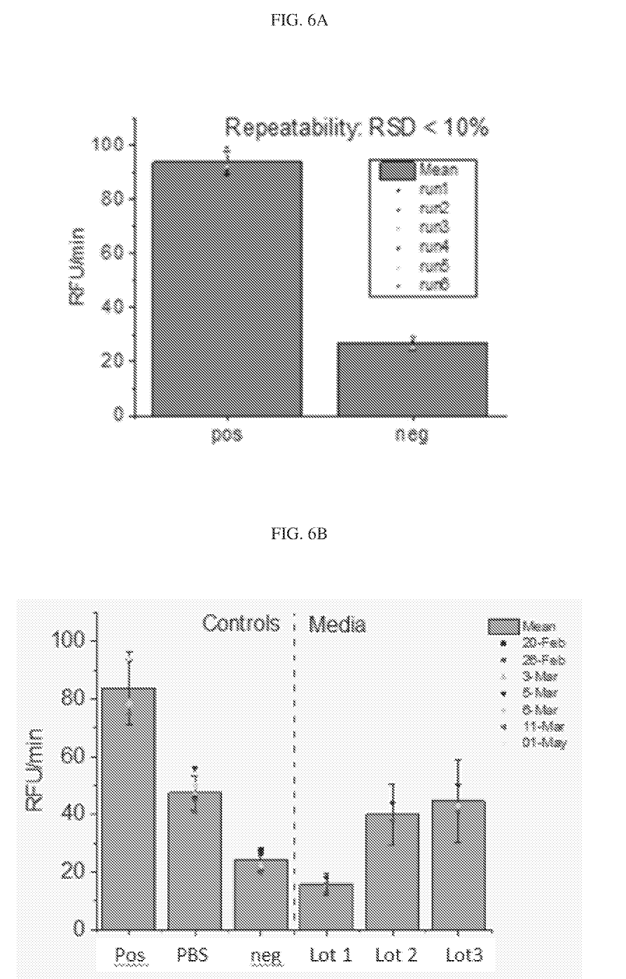

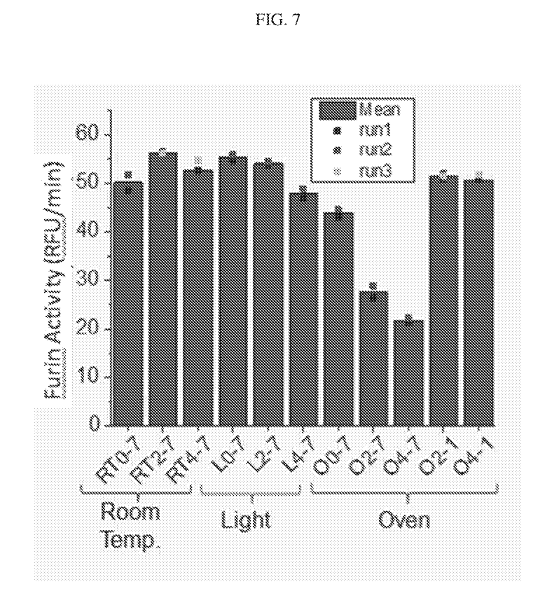

Furin activity was tested on different dates to measure precision (FIGS. 6A-6B).

Degradation factors include time, light, temperature and moisture content. Inhibition (RFU/min) values of a LO NPI OptiCHO™ Lot as a function of various degradation factors including room temperature (no light), light (1 W/m²) and an oven (˜40° C.) were obtained (FIG. 7).

Furin activity correlates with low and high NPI lots of OptiCHO™. Preliminary studies have predicted current low NPI results, however it is not confirmed if the correlation is causal. Assay characteristics include intermediate precision (˜10% in PBS and ˜20% in medium). The repeatability is 10%. The sensitivity of the assay, R², is 0.9 (NPI vs. RFU/min). The range was tested with other medium and Furin activity was not saturated. PBS can be used as an assay control. As a negative control, low NPI lots can be degraded with heat to inhibit furin activity.

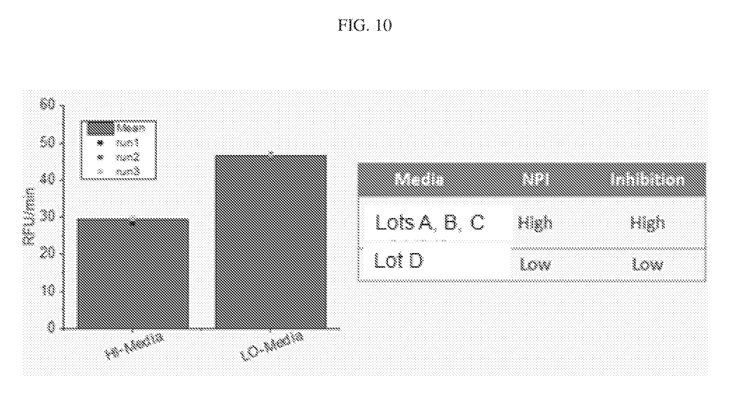

In some embodiments, the presence of inhibitors can also be detected in spent samples or media (see, e.g., FIG. 10).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

SEQUENCES
PCSK1 protein sequence SEQ ID NO: 1 (gi|20336242|ref|
NP_000430.3)
MERRAWSLQCTAFVLFCAWCALNSAKAKRQFVNEWAAEIPGGPEAASAIAEELGYD
LLGQIGSLENHYLFKHKNHPRRSRRSAFHITKRLSDDDRVIWAEQQYEKERSKRSAL
RDSALNLFNDPMWNQQWYLQDTRMTAALPKLDLHVIPVWQKGITGKGVVITVLDD
GLEWNHTDIYANYDPEASYDFNDNDHDPFPRYDPTNENKHGTRCAGEIAMQANNH
KCGVGVAYNSKVGGIRMLDGIVTDAIEASSIGFNPGHVDIYSASWGPNDDGKTVEGP
GRLAQKAFEYGVKQGRQGKGSIFVWASGNGGRQGDNCDCDGYTDSIYTISISSASQ
QGLSPWYAEKCSSTLATSYSSGDYTDQRITSADLHNDCTETHTGTSASAPLAAGIFAL
ALEANPNLTWRDMQHLVVWTSEYDPLANNPGWKKNGAGLMVNSRFGFGLLNAKA
LVDLADPRTWRSVPEKKECVVKDNDFEPRALKANGEVIIEIPTRACEGQENAIKSLEH
VQFEATIEYSRRGDLHVTLTSAAGTSTVLLAERERDTSPNGFKNWDFMSVHTWGEN
PIGTWTLRITDMSGRIQNEGRIVNWKLILHGTSSQPEHMKQPRVYTSYNTVQNDRRG
VEKMVDPGEEQPTQENPKENTLVSKSPSSSSVGGRRDELEEGAPSQAMLRLLQSAFS
KNSPPKQSPKKSPSAKLNIPYENFYEALEKLNKPSQLKDSEDSLYNDYVDVFYNTKP
YKHRDDRLLQALVDILNEEN
PCSK2 protein sequence SEQ ID NO: 2 (gi|320118926|ref|
NP_001188457.1)
MVFASAERPVFTNHFLVELHKGGEDKARQVAAEHGFGVRKLPFAEGLYHFYHNGL
AKAKRRRSLHHKQQLERDPRVKMALQQEGFDRKKRGYRDINEIDINMNDPLFTKQ
WYLINTGQADGTPGLDLNVAEAWELGYTGKGVTIGIMDDGIDYLHPDLASNYNAEA
SYDFSSNDPYPYPRYTDDWFNSHGTRCAGEVSAAANNNICGVGVAYNSKVAGIRML
DQPFMTDIIEASSISHMPQLIDIYSASWGPTDNGKTVDGPRELTLQAMADGVNKGRG
GKGSIYVWASGDGGSYDDCNCDGYASSMWTISINSAINDGRTALYDESCSSTLASTF
SNGRKRNPEAGVATTDLYGNCTLRHSGTSAAAPEAAGVFALALEANLGLTWRDMQ
HLTVLTSKRNQLHDEVHQWRRNGVGLEFNHLFGYGVLDAGAMVKMAKDWKTVPE
RFHCVGGSVQDPEKIPSTGKLVLTLTTDACEGKENFVRYLEHVQAVITVNATRRGDL
NINMTSPMGTKSILLSRRPRDDDSKVGFDKWPFMTTHTWGEDARGTWTLELGFVGS
APQKGVLKEWTLMLHGTQSAPYIDQVVRDYQSKLAMSKKEELEEELDEAVERSLKS
ILNKN
PCSK3 protein sequence SEQ ID NO: 3 (gi|577019578|ref|
NP_001276752.1)
MELRPWLLWVVAATGTLVLLAADAQGQKVFTNTWAVRIPGGPAVANSVARKHGF
LNLGQIFGDYYHFWHRGVTKRSLSPHRPRHSRLQREPQVQWLEQQVAKRRTKRDV
YQEPTDPKFPQQWYLSGVTQRDLNVKAAWAQGYTGHGIVVSILDDGIEKNHPDLAG
NYDPGASFDVNDQDPDPQPRYTQMNDNRHGTRCAGEVAAVANNGVCGVGVAYNA
RIGGVRMLDGEVTDAVEARSLGLNPNHIHIYSASWGPEDDGKTVDGPARLAEEAFFR
GVSQGRGGLGSIFVWASGNGGREHDSCNCDGYTNSIYTLSISSATQFGNVPWYSEAC
SSTLATTYSSGNQNEKQIVTTDLRQKCTESHTGTSASAPLAAGIIALTLEANKNLTWR
DMQHLVVQTSKPAHLNANDWATNGVGRKVSHSYGYGLLDAGAMVALAQNWTTV
APQRKCIIDILTEPKDIGKRLEVRKTVTACLGEPNHITRLEHAQARLTLSYNRRGDLAI
HLVSPMGTRSTLLAARPHDYSADGFNDWAFMTTHSWDEDPSGEWVLEIENTSEANN
YGTLTKFTLVLYGTAPEGLPVPPESSGCKTLTSSQACVVCEEGFSLHQKSCVQHCPPG
FAPQVLDTHYSTENDVETIRASVCAPCHASCATCQGPALTDCLSCPSHASLDPVEQT
CSRQSQSSRESPPQQQPPRLPPEVEAGQRLRAGLLPSHLPEVVAGLSCAFIVLVFVTVF
LVLQLRSGFSFRGVKVYTMDRGLISYKGLPPEAWQEECPSDSEEDEGRGERTAFIKD
QSAL
PCSK4 protein sequence SEQ ID NO: 4 (gi|76443679|ref|
NP_060043.2)
MRPAPIALWLRLVLALALVRPRAVGWAPVRAPIYVSSWAVQVSQGNREVERLARKF
GFVNLGPIFPDGQYFHLRHRGVVQQSLTPHWGHRLHLKKNPKVQWFQQQTLQRRV
KRSVVVPTDPWFSKQWYMNSEAQPDLSILQAWSQGLSGQGIVVSVLDDGIEKDHPD
LWANYDPLASYDFNDYDPDPQPRYTPSKENRHGTRCAGEVAAMANNGFCGVGVAF
NARIGGVRMLDGTITDVIEAQSLSLQPQHIHIYSASWGPEDDGRTVDGPGILTREAFR
RGVTKGRGGLGTLFIWASGNGGLHYDNCNCDGYTNSIHTLSVGSTTQQGRVPWYSE
ACASTLTTTYSSGVATDPQIVTTDLHHGCTDQHTGTSASAPLAAGMIALALEANPFL
TWRDMQHLVVRASKPAHLQAEDWRTNGVGRQVSHHYGYGLLDAGLLVDTARTW
LPTQPQRKCAVRVQSRPTPILPLIYIRENVSACAGLHNSIRSLEHVQAQLTLSYSRRGD
LEISLTSPMGTRSTLVAIRPLDVSTEGYNNWVFMSTHFWDENPQGVWTLGLENKGY
YFNTGTLYRYTLLLYGTAEDMTARPTGPQVTSSACVQRDTEGLCQACDGPAYILGQ
LCLAYCPPRFFNHTRLVTAGPGHTAAPALRVCSSCHASCYTCRGGSPRDCTSCPPSST
LDQQQGSCMGPTTPDSRPRLRAAACPHHRCPASAMVLSLLAVTLGGPVLCGMSMD
LPLYAWLSRARATPTKPQVWLPAGT
PCSK5 protein sequence SEQ ID NO: 5 (gi|20336246|ref|
NP_006191.2)
MGWGSRCCCPGRLDLLCVLALLGGCLLPVCRTRVYTNHWAVKIAGGFPEANRIASK
YGFINIGQIGALKDYYHFYHSRTIKRSVISSRGTHSFISMEPKVEWIQQQVVKKRTKR
DYDFSRAQSTYFNDPKWPSMWYMHCSDNTHPCQSDMNIEGAWKRGYTGKNIVVTI
LDDGIERTHPDLMQNYDALASCDVNGNDLDPMPRYDASNENKHGTRCAGEVAAAA
NNSHCTVGIAFNAKIGGVRMLDGDVTDMVEAKSVSFNPQHVHIYSASWGPDDDGK
TVDGPAPLTRQAFENGVRMGRRGLGSVFVWASGNGGRSKDHCSCDGYTNSIYTISIS
STAESGKKPWYLEECSSTLATTYSSGESYDKKIITTDLRQRCTDNHTGTSASAPMAA
GIIALALEANPFLTWRDVQHVIVRTSRAGHLNANDWKTNAAGFKVSHLYGFGLMDA
EAMVMEAEKWTTVPRQHVCVESTDRQIKTIRPNSAVRSIYKASGCSDNPNRHVNYL
EHVVVRITITHPRRGDLAIYLTSPSGTRSQLLANRLFDHSMEGFKNWEFMTIHCWGE
RAAGDWVLEVYDTPSQLRNFKTPGKLKEWSLVLYGTSVQPYSPTNEFPKVERFRYS
RVEDPTDDYGTEDYAGPCDPECSEVGCDGPGPDHCNDCLHYYYKLKNNTRICVSSC
PPGHYHADKKRCRKCAPNCESCFGSHGDQCMSCKYGYFLNEETNSCVTHCPDGSYQ
DTKKNLCRKCSENCKTCTEFHNCTECRDGLSLQGSRCSVSCEDGRYFNGQDCQPCH
RFCATCAGAGADGCINCTEGYFMEDGRCVQSCSISYYFDHSSENGYKSCKKCDISCL
TCNGPGFKNCTSCPSGYLLDLGMCQMGAICKDATEESWAEGGFCMLVKKNNLCQR
KVLQQLCCKTCTFQG
PCSK6 protein sequence SEQ ID NO: 6 (gi|604723363|ref|
NP_001278238.1)
MPPRAPPAPGPRPPPRAAAATDTAAGAGGAGGAGGAGGPGFRPLAPRPWRWLLLL
ALPAACSAPPPRPVYTNHWAVQVLGGPAEADRVAAAHGYLNLGQIGNLEDYYHFY
HSKTFKRSTLSSRGPHTFLRMDPQVKWLQQQEVKRRVKRQVRSDPQALYFNDPIWS
NMWYLHCGDKNSRCRSEMNVQAAWKRGYTGKNVVVTILDDGIERNHPDLAPNYD
SYASYDVNGNDYDPSPRYDASNENKHGTRCAGEVAASANNSYCIVGIAYNAKIGGI
RMLDGDVTDVVEAKSLGIRPNYIDIYSASWGPDDDGKTVDGPGRLAKQAFEYGIKK
GRQGLGSIFVWASGNGGREGDYCSCDGYTNSIYTISVSSATENGYKPWYLEECASTL
ATTYSSGAFYERKIVTTDLRQRCTDGHTGTSVSAPMVAGIIALALEAKSIPLVQVLRT
TALTSACAEHSDQRVVYLEHVVVRTSISHPRRGDLQIYLVSPSGTKSQLLAKRLLDLS
NEGFTNWEFMTVHCWGEKAEGQWTLEIQDLPSQVRNPEKQGKLKEWSLILYGTAE
HPYHTFSAHQSRSRMLELSAPELEPPKAALSPSQVEVPEDEEDYTAQSTPGSANILQT
SVCHPECGDKGCDGPNADQCLNCVHFSLGSVKTSRKCVSVCPLGYFGDTAARRCRR
CHKGCETCSSRAATQCLSCRRGFYHHQEMNTCVTLCPAGFYADESQKNCLKCHPSC
KKCVDEPEKCTVCKEGFSLARGSCIPDCEPGTYFDSELIRCGECHHTCGTCVGPGREE
CIHCAKNFHFHDWKCVPACGEGFYPEEMPGLPHKVCRRCDENCLSCAGSSRNCSRC
KTGFTQLGTSCITNHTCSNADETFCEMVKSNRLCERKLFIQFCCRTCLLAG
PCSK7 protein sequence SEQ ID NO: 7 (gi|20336248|ref|
NP_004707.2)
MPKGRQKVPHLDAPLGLPTCLWLELAGLFLLVPWVMGLAGTGGPDGQGTGGPSWA
VHLESLEGDGEEETLEQQADALAQAAGLVNAGRIGELQGHYLFVQPAGHRPALEVE
AIRQQVEAVLAGHEAVRWHSEQRLLRRAKRSVHFNDPKYPQQWHLNNRRSPGRDI
NVTGVWERNVTGRGVTVVVVDDGVEHTIQDIAPNYSPEGSYDLNSNDPDPMPHPDV
ENGNHHGTRCAGEIAAVPNNSFCAVGVAYGSRIAGIRVLDGPLTDSMEAVAFNKHY
QINDIYSCSWGPDDDGKTVDGPHQLGKAALQHGVIAGRQGFGSIFVVASGNGGQHN
DNCNYDGYANSIYTVTIGAVDEEGRMPFYAEECASMLAVTFSGGDKMLRSIVTTDW
DLQKGTGCTEGHTGTSAAAPLAAGMIALMLQVRPCLTWRDVQHIIVFTATRYEDRR
AEWVTNEAGFSHSHQHGFGLLNAWRLVNAAKIWTSVPYLASYVSPVLKENKAIPQS
PRSLEVLWNVSRMDLEMSGLKTLEHVAVTVSITHPRRGSLELKLFCPSGMMSLIGAP
RSMDSDPNGFNDWTFSTVRCWGERARGTYRLVIRDVGDESFQVGILRQWQLTLYGS
VWSAVDIRDRQRLLESAMSGKYLHDDFALPCPPGLKIPEEDGYTITPNTLKTLVLVG
CFTVFWTVYYMLEVYLSQRNVASNQVCRSGPCHWPHRSRKAKEEGTELESVPLCSS
KDPDEVETESRGPPTTSDLLAPDLLEQGDWSLSQNKSALDCPHQHLDVPHGKEEQIC
PCSK8 protein sequence SEQ ID NO: 8 (gi|4506775|ref|
NP_003782.1)
MKLVNIWLLLLVVLLCGKKHLGDRLEKKSFEKAPCPGCSHLTLKVEFSSTVVEYEYI
VAFNGYFTAKARNSFISSALKSSEVDNWRIIPRNNPSSDYPSDFEVIQIKEKQKAGLLT
LEDHPNIKRVTPQRKVFRSLKYAESDPTVPCNETRWSQKWQSSRPLRRASLSLGSGF
WHATGRHSSRRLLRAIPRQVAQTLQADVLWQMGYTGANVRVAVFDTGLSEKHPHF
KNVKERTNWTNERTLDDGLGHGTFVAGVIASMRECQGFAPDAELHIFRVFTNNQVS
YTSWFLDAFNYAILKKIDVLNLSIGGPDFMDHPFVDKVWELTANNVIMVSAIGNDGP
LYGTLNNPADQMDVIGVGGIDFEDNIARFSSRGMTTWELPGGYGRMKPDIVTYGAG
VRGSGVKGGCRALSGTSVASPVVAGAVTLLVSTVQKRELVNPASMKQALIASARRL
PGVNMFEQGHGKLDLLRAYQILNSYKPQASLSPSYIDLTECPYMWPYCSQPIYYGGM
PTVVNVTILNGMGVTGRIVDKPDWQPYLPQNGDNIEVAFSYSSVLWPWSGYLAISIS
VTKKAASWEGIAQGHVMITVASPAETESKNGAEQTSTVKLPIKVKIIPTPPRSKRVLW
DQYHNLRYPPGYFPRDNLRMKNDPLDWNGDHIHTNFRDMYQHLRSMGYFVEVLG
APFTCFDASQYGTLLMVDSEEEYFPEEIAKLRRDVDNGLSLVIFSDWYNTSVMRKVK
FYDENTRQWWMPDTGGANIPALNELLSVWNMGFSDGLYEGEFTLANHDMYYASG
CSIAKFPEDGVVITQTFKDQGLEVLKQETAVVENVPILGLYQIPAEGGGRIVLYGDSN
CLDDSHRQKDCFWLLDALLQYTSYGVTPPSLSHSGNRQRPPSGAGSVTPERMEGNH
LHRYSKVLEAHLGDPKPRPLPACPRLSWAKPQPLNETAPSNLWKHQKLLSIDLDKVV
LPNFRSNRPQVRPLSPGESGAWDIPGGIMPGRYNQEVGQTIPVFAFLGAMVVLAFFV
VQINKAKSRPKRRKPRVKRPQLMQQVHPPKTPSV
PCSK9 protein sequence SEQ ID NO: 9 (gi|31317307|ref|
NP_777596.2)
MGTVSSRRSWWPLPLLLLLLLLLGPAGARAQEDEDGDYEELVLALRSEEDGLAEAP
EHGTTATFHRCAKDPWRLPGTYVVVLKEETHLSQSERTARRLQAQAARRGYLTKIL
HVFHGLLPGFLVKMSGDLLELALKLPHVDYIEEDSSVFAQSIPWNLERITPPRYRADE
YQPPDGGSLVEVYLLDTSIQSDHREIEGRVMVTDFENVPEEDGTRFHRQASKCDSHG
THLAGVVSGRDAGVAKGASMRSLRVLNCQGKGTVSGTLIGLEFIRKSQLVQPVGPL
VVLLPLAGGYSRVLNAACQRLARAGVVLVTAAGNFRDDACLYSPASAPEVITVGAT
NAQDQPVTLGTLGTNFGRCVDLFAPGEDIIGASSDCSTCFVSQSGTSQAAAHVAGIA
AMMLSAEPELTLAELRQRLIHFSAKDVINEAWFPEDQRVLTPNLVAALPPSTHGAGW
QLFCRTVWSAHSGPTRMATAVARCAPDEELLSCSSFSRSGKRRGERMEAQGGKLVC
RAHNAFGGEGVYAIARCCLLPQANCSVHTAPPAEASMGTRVHCHQQGHVLTGCSSH
WEVEDLGTHKPPVLRPRGQPNQCVGHREASIHASCCHAPGLECKVKEHGIPAPQEQ
VTVACEEGWTLTGCSALPGTSHVLGAYAVDNTCVVRSRDVSTTGSTSEGAVTAVAI
CCRSRHLAQASQELQ
Subtilisin E protein sequence from Bacillus subtilis,
SEQ ID NO: 10
MRSKKLWISLLFALTLIFTMAFSNMSAQAAGKSSTEKKYIVGFKQTMSAMSSAKKK
DVISEKGGKVQKQFKYVNAAAATLDEKAVKELKKDPSVAYVEEDHIAHEYAQSVP
YGISQIKAPALHSQGYTGSNVKVAVIDSGIDSSHPDLNVRGGASFVPSETNPYQDGSS
HGTHVAGTIAALNNSIGVLGVAPSASLYAVKVLDSTGSGQYSWIINGIEWAISNNMD
VINMSLGGPTGSTALKTVVDKAVSSGIVVAAAAGNEGSSGSTSTVGYPAKYPSTIAV
GAVNSSNQRASFSSAGSELDVMAPGVSIQSTLPGGTYGAYNGTSMATPHVAGAAAL
ILSKHPTWTNAQVRDRLESTATYLGNSFYYGKGLINVQAAAQ
KEX2 protein sequence from Saccharomyces
cerevisiae, SEQ ID NO: 11 (gi|6324091|ref|NP_014161.1)
MKVRKYITLCFWWAFSTSALVSSQQIPLKDHTSRQYFAVESNETLSRLEEMHPNWK
YEHDVRGLPNHYVFSKELLKLGKRSSLEELQGDNNDHILSVHDLFPRNDLFKRLPVP
APPMDSSLLPVKEAEDKLSINDPLFERQWHLVNPSFPGSDINVLDLWYNNITGAGVV
AAIVDDGLDYENEDLKDNFCAEGSWDFNDNTNLPKPRLSDDYHGTRCAGEIAAKKG
NNFCGVGVGYNAKISGIRILSGDITTEDEAASLIYGLDVNDIYSCSWGPADDGRHLQG
PSDLVKKALVKGVTEGRDSKGAIYVFASGNGGTRGDNCNYDGYTNSIYSITIGAIDH
KDLHPPYSEGCSAVMAVTYSSGSGEYIHSSDINGRCSNSHGGTSAAAPLAAGVYTLL
LEANPNLTWRDVQYLSILSAVGLEKNADGDWRDSAMGKKYSHRYGFGKIDAHKLIE
MSKTWENVNAQTWFYLPTLYVSQSTNSTEETLESVITISEKSLQDANFKRIEHVTVTV
DIDTEIRGTTTVDLISPAGIISNLGVVRPRDVSSEGFKDWTFMSVAHWGENGVGDWKI
KVKTTENGHRIDFHSWRLKLFGESIDSSKTETFVFGNDKEEVEPAATESTVSQYSASS
TSISISATSTSSISIGVETSAIPQTTTASTDPDSDPNTPKKLSSPRQAMHYFLTIFLIGATF
LVLYFMFFMKSRRRIRRSRAETYEFDIIDTDSEYDSTLDNGTSGITEPEEVEDFDFDLS
DEDHLASLSSSENGDAEHTIDSVLTNENPFSDPIKQKFPNDANAESASNKLQELQPDV
PPSSGRS

What is claimed is:

1. A method of promoting recombinant protein yield from a recombinant cell culture in a bioreactor, the method comprising
performing an assay to determine a level of an inhibitor of proprotein convertase activity in one or more cell culture reagents, and
using a cell culture reagent to support growth of the recombinant cell culture in the bioreactor only if the cell culture reagent is determined to contain a proprotein convertase inhibitor in an amount that is acceptable for the recombinant protein yield.

2. The method of claim 1, wherein the assay is a proprotein convertase substrate assay comprising:
(a) combining a sample of reagent with
(i) a probe that comprises a proprotein convertase recognition site and emits a detectable signal when cleaved at the recognition site, and
(ii) a cognate proprotein convertase, thereby forming a mixture; and
(b) incubating the mixture under conditions that result in cleavage of the probe at the proprotein convertase recognition site.

3. The method of claim 2 further comprising performing a signal detection assay on the mixture.

4. The method of claim 3 further comprising detecting a signal in the mixture as a result of performing the signal detection assay, wherein the amount of signal detected in the mixture is proportional to the amount of activity of the cognate proprotein convertase in the sample of the reagent.

5. The method of any one of claims 2-4, wherein the detectable signal is a fluorescent signal.

6. The method of any one of claims 2-5, wherein the proprotein convertase recognition site comprises 2 or more amino acids of a PACE cleavage site.

7. The method of any one of claims 2-6, wherein the proprotein convertase recognition site comprises the following amino acid consensus sequence: R-X-X-R, wherein R is arginine and X is any amino acid.

8. The method of claim 7, wherein the proprotein convertase recognition site comprises the following amino acid consensus sequence: R-X-(K/R)-R (SEQ ID NO: 12), wherein R is arginine, X is any amino acid, and K is lysine.

9. The method of claim 8, wherein the proprotein convertase recognition site comprises the following amino acid sequence: R-V-R-R (SEQ ID NO: 13), wherein R is arginine, and V is valine.

10. The method of any one of claims 1-9, wherein the proprotein convertase is selected from the group consisting of: PCSK1, PCSK2, PCSK3/furin, PCSK4, PCSK5, PCSK6, PCSK7 and Kex2.

11. The method of claim 10, wherein the proprotein convertase is PCSK3/furin.

12. The method of any one of claims 1-11, wherein the reagent is a powdered or liquid cell culture medium.

13. The method of any one of claims 2-12, wherein the sample of the reagent is cell-free.

14. The method of any one of claims 2-13, wherein the sample of the reagent is protein-free.

15. The method of any one of claims 2-14, wherein the probe comprises a protecting group linked to a fluorophore via a linker comprising the proprotein convertase recognition site.

16. The method of claim 15, wherein the protecting group is a tert-butyloxycarbonyl (t-Boc) protecting group.

17. The method of claim 15, wherein the protecting group is a 9-fluorenylmethyloxycarbonyl (Fmoc) protecting group.

18. The method of claim 15, wherein the fluorophore is 7-Amino-4-methylcoumarin (AMC).

19. The method of claim 18, wherein the probe comprises a t-Boc protecting group linked to AMC via a linker that comprises the following amino acid sequence: R-V-R-R (SEQ ID NO: 13), wherein R is arginine, and V is valine.

20. The method of claim 4 further comprising comparing the signal detected in the mixture to a signal detected in a separate mixture produced by
(c) combining a sample of reagent with
(i) the probe that comprises the proprotein convertase recognition site and emits a detectable signal when cleaved at the recognition site, and
(ii) the cognate proprotein convertase, thereby producing the separate mixture; and
(d) incubating the separate mixture produced in step (c)(ii) under conditions that result in cleavage of the probe of step (c)(i).

21. The method of claim 20, wherein the sample of the reagent of (a) and the sample of the reagent of (c) are of the same type and are obtained from separate lots.

22. The method of claim 20 or 21 further comprising performing a signal detection assay on the separate mixture.

23. The method of claim 22 further comprising detecting a signal in the separate mixture as a result of performing the signal detection assay, wherein the amount of signal detected in the separate mixture is proportional to the amount of activity of the cognate proprotein convertase in the sample of reagent used in step (c).

24. The method of claim 23 further comprising selecting the sample of reagent used in the mixture produced in (a)(ii) or selecting the sample of reagent used in the separate mixture produced in (c)(ii) based on the amount of signal detected in each of the mixtures.

25. A method of selecting a reagent for use in the production of a recombinant protein, the method comprising;
performing a proprotein convertase substrate assay in the presence of a sample of a reagent;
determining a level of activity of a proprotein convertase; and
identifying the reagent as acceptable for use in a recombinant cell culture to produce the recombinant protein if the level of activity of the proprotein convertase is at or above a threshold level sufficient for recombinant protein production.

26. The method of claim 24, wherein the level of activity of the proprotein convertase is determined relative to the level of activity of a second proprotein convertase in the presence of a second sample of reagent that does not inhibit the activity of the second proprotein convertase.

27. The method of claim 26, wherein the threshold level is the level of activity of the second proprotein convertase.

28. The method of claim 26, wherein the threshold level is 95%, 90%, 85%, 80%, 70%, 60%, 50%, 40%, 30%, 20%, or 10% of the level of activity of the second proprotein convertase.
